SEQUENCE LISTING (22 sequences)

SEQ ID NO: 1 - RNA, 1621 nt, Homo sapiens
  source 1..1621: mol_type = mRNA
agcagggctcccgcgggcgtcactccggccatcgtccccacctccacctgggccgcccggcaggcaggcgacggaggcccgggagccatgggtgactggggcttcctggagaagttgctggaccaggtccaggagcactcgaccgtggtgggtaagatctggctgacggtgctcttcatcttccgcatcctcatcctgggcctggccggcgagtcagtgtggggtgacgagcaatcagatttcgagtgtaacacggcccagccaggctgcaccaacgtctgctatgaccaggccttccccatctcccacatccgctactgggtgctgcagttcctcttcgtcagcacacccaccctggtctacctgggccatgtcatttacctgtctcggcgagaagagcggctgcggcagaaggagggggagctgcgggcactgccggccaaggacccacaggtggagcgggcgctggcggccgtagagcgtcagatggccaagatctcggtggcagaagatggtcgcctgcgcatccgcggagcactgatgggcacctatgtcgccagtgtgctctgcaagagtgtgctagaggcaggcttcctctatggccagtggcgcctgtacggctggaccatggagcccgtgtttgtgtgccagcgagcaccctgcccctacctcgtggactgctttgtctctcgccccacggagaagaccatcttcatcatcttcatgttggtggttggactcatctccctggtgcttaacctgctggagttggtgcacctgctgtgtcgctgcctcagccgggggatgagggcacggcaaggccaagacgcacccccgacccagggcacctcctcagacccttacacggaccaggtcttcttctacctccccgtgggccaggggccctcatccccaccatgccccacctacaatgggctctcatccagtgagcagaactgggccaacctgaccacagaggagaggctggcgtcttccaggccccctctcttcctggacccaccccctcagaatggccaaaaacccccaagtcgtcccagcagctctgcttctaagaagcagtatgtatagaggcctgtggcttatgtcacccaacagaggggtcctgagaagtctggctgcctgggatgccccctgccccctcctggaaggctctgcagagatgactgggctggggaagcaggtgcttgctggccatggagcctcattgcaagttgttcttgaacacctgaggccttcctggtgcccaccaggcactacggcttcctctccagaatgtggctttgcctgagcacagacagagtcagcatggaatgctcttggccaagggtactgggggccctctggccttttgcagctgatccagaggaacccagagccaacttaccccaacctcaccctatggaacagtcacctgtgcgcaggttgtcctcaaaccctctcctcacaggaaaaggcggattgaggctgctgggtcagccttgatcgcacagacagagcttgtgccggatttggccctgtcaaggggactggtgccttgttttcatcactccttcctagttctactgttcaagcttctgaaataaacaggacttgatcacaa

SEQ ID NO: 2 - protein, 333 aa, Homo sapiens
MGDWGFLEKLLDQVQEHSTVVGKIWLTVLFIFRILILGLAGESVWGDEQSDFECNTAQPGCTNVCYDQAFPISHIRYWVLQFLFVSTPTLVYLGHVIYLSRREERLRQKEGELRALPAKDPQVERALAAVERQMAKISVAEDGRLRIRGALMGTYVASVLCKSVLEAGFLYGQWRLYGWTMEPVFVCQRAPCPYLVDCFVSRPTEKTIFIIFMLVVGLISLVLNLLELVHLLCRCLSRGMRARQGQDAPPTQGTSSDPYTDQVFFYLPVGQGPSSPPCPTYNGLSSSEQNWANLTTEERLASSRPPLFLDPPPQNGQKPPSRPSSSASKKQYV

SEQ ID NO: 3 - RNA, 3083 nt, Homo sapiens
  source 1..3083: mol_type = mRNA
aaaagcttttacgaggtatcagcacttttctttcattagggggaaggcgtgaggaaagtaccaaacagcagcggagttttaaactttaaatagacaggtctgagtgcctgaacttgccttttcattttacttcatcctccaaggagttcaatcacttggcgtgacttcactacttttaagcaaaagagtggtgcccaggcaacatgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctagatacaggcttgaaagcatcaagattccactcaattgtggagaagaaaaaaggtgctgtagaaagtgcaccaggtgttaattttgatccggtggaggtggtactcaacagccttattcatgaggcttagaaaacacaaagacattagaatacctaggttcactgggggtgtatggggtagatgggtggagagggaggggataagagaggtgcatgttggtatttaaagtagtggattcaaagaacttagattataaataagagttccattaggtgatacatagataagggctttttctccccgcaaacacccctaagaatggttctgtgtatgtgaatgagcgggtggtaattgtggctaaatatttttgttttaccaagaaactgaaataattctggccaggaataaatacttcctgaacatcttaggtcttttcaacaagaaaaagacagaggattgtccttaagtccctgctaaaacattccattgttaaaatttgcactttgaaggtaagctttctaggcctgaccctccaggtgtcaatggacttgtgctactatatttttttattcttggtatcagtttaaaattcagacaaggcccacagaataagattttccatgcatttgcaaatacgtatattctttttccatccacttgcacaatatcattaccatcactttttcatcattcctcagctactactcacattcatttaatggtttctgtaaacatttttaagacagttgggatgtcacttaacattttttttttgagctaaagtcagggaatcaagccatgcttaatatttaacaatcacttatatgtgtgtcgaagagtttgttttgtttgtcatgtattggtacaagcagatacagtataaactcacaaacacagatttgaaaataatgcacatatggtgttcaaatttgaacctttctcatggatttttgtggtgtgggccaatatggtgtttacattatataattcctgctgtggcaagtaaagcacactttttttttctcctaaaatgtttttccctgtgtatcctattatggatactggttttgttaattatgattctttattttctctcctttttttaggatatagcagtaatgctattactgaaatgaatttcctttttctgaaatgtaatcattgatgcttgaatgatagaattttagtactgtaaacaggctttagtcattaatgtgagagacttagaaaaaatgcttagagtggactattaaatgtgcctaaatgaattttgcagtaactggtattcttgggttttcctacttaatacacagtaattcagaacttgtattctattatgagtttagcagtcttttggagtgaccagcaactttgatgtttgcactaagattttatttggaatgcaagagaggttgaaagaggattcagtagtacacatacaactaatttatttgaactatatgttgaagacatctaccagtttctccaaatgccttttttaaaactcatcacagaagattggtgaaaatgctgagtatgacacttttcttcttgcatgcatgtcagctacataaacagttttgtacaatgaaaattactaatttgtttgacattccatgttaaactacggtcatgttcagcttcattgcatgtaatgtagacctagtccatcagatcatgtgttctggagagtgttctttattcaataaagttttaatttagtataaacata

SEQ ID NO: 4 - protein, 382 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRPDDLEI

SEQ ID NO: 5 - DNA, 2706 bp, Homo sapiens
  source 1..2706: mol_type = genomic DNA
ctggccgcagtcgcggcagtggtggcttcccatccccaaaaggcgccctccgactccttgcgccgcactgctcgccgggccagtccggaaacgggtcgtggagctccgcaccactcccgctggttcccgaaggcagatcccttctcccgagagttgcgagaaactttcccttgtccccgacgctgcagcggctcgggtaccgtggcagccgcaggtttctgaaccccgggccacgctccccgcgcctcggcttcgcgctcgtgtagatcgttccctctctggttgcacgctggggatcccggacctcgattctgcgggcgagatgcccctgggacacatcatgaggctggacctggagaaaattgccctggagtacatcgtgccctgtctgcacgaggtgggcttctgctacctggacaacttcctgggcgaggtggtgggcgactgcgtcctggagcgcgtcaagcagctgcactgcaccggggccctgcgggacggccagctggcggggccgcgcgccggcgtctccaagcgacacctgcggggcgaccagatcacgtggatcgggggcaacgaggagggctgcgaggccatcagcttcctcctgtccctcatcgacaggctggtcctctactgcgggagccggctgggcaaatactacgtcaaggagaggtctaaggcaatggtggcttgctatccgggaaatggaacaggttatgttcgccacgtggacaaccccaacggtgatggtcgctgcatcacctgcatctactatctgaacaagaattgggatgccaagctacatggtgggatcctgcggatatttccagaggggaaatcattcatagcagatgtggagcccatttttgacagactcctgttcttctggtcagatcgtaggaacccacacgaagtgcagccctcttacgcaaccagatatgctatgactgtctggtactttgatgctgaagaaagggcagaagccaaaaagaaattcaggaatttaactaggaaaactgaatctgccctcactgaagactgaccgtgctctgaaatctgctggccttgttcattttagtaacggttcctgaattctcttaaattctttgagatccaaagatggcctcttcagtgacaacaatctccctgctacttcttgcatccttcacatccctgtcttgtgtgtggtacttcatgttttcttgccaagactgtgttgatcttcagatactctctttgccagatgaagttacttgctaactccagaaattcctgcagacatcctactcggccagcggtttacctgatagattcggtaatactatcaagagaagagcctaggagcacagcgagggaatgaaccttacttgcactttatgtatacttcctgatttgaaaggaggaggtttgaaaagaaaaaaatggaggtggtagatgccacagagaggcatcacggaagccttaacagcaggaaacagagaaatttgtgtcatctgaacaatttccagatgttcttaatccagggctgttggggtttctggagaattatcacaacctaatgacattaatacctctagaaagggctgctgtcatagtgaacaatttataagtgtcccatggggcagacactccttttttcccagtcctgcaacctggattttctgcctcagccccattttgctgaaaataatgactttctgaataaagatggcaacacaattttttctccattttcagttcttacctgggaacctaattccccagaagctaaaaaactagacattagttgttttggttgctttgttggaatggaatttaaatttaaatgaaaggaaaaatatatccctggtagttttgtgttaaccactgataactgtggaaagagctaggtctactgatatacaataaacatgtgtgcatcttgaacaatttgagaggggaggtggagttggaaatgtgggtgttcctgttttttttttttttttttttttagttttcctttttaatgagctcaccctttaacacaaaaaaagcaaggtgatgtattttaaaaaaggaagtggaaataaaaaaatctcaaagctatttgagttctcgtctgtccctagcagtctttcttcagctcacttggctctctagatccactgtggttggcagtatgaccagaatcatggaatttgctagaactgtggaagcttctactcctgcagtaagcacagatcgcactgcctcaataacttggtattgagcacgtattttgcaaaagctacttttcctagttttcagtattactttcatgttttaaaaatccctttaatttcttgcttgaaaatcccatgaacattaaagagccagaaatattttcctttgttatgtacggatatatatatatatagtcttccaagatagaagtttactttttcctcttctggttttggaaaatttccagataagacatgtcaccattaattctcaacgactgctctattttgttgtacggtaatagttatcaccttctaaattactatgtaatttattcacttattatgtttattgtcttgtatcctttctctggagtgtaagcacaatgaagacaggaattttgtatatttttaaccaatgcaacatactctcagcacctaaaatagtgccgggaacatagtaagggctcagtaaatacttgttgaataaactcagtctcctaca

SEQ ID NO: 6 - protein, 239 aa, Homo sapiens
MPLGHIMRLDLEKIALEYIVPCLHEVGFCYLDNFLGEVVGDCVLERVKQLHCTGALRDGQLAGPRAGVSKRHLRGDQITWIGGNEEGCEAISFLLSLIDRLVLYCGSRLGKYYVKERSKAMVACYPGNGTGYVRHVDNPNGDGRCITCIYYLNKNWDAKLHGGILRIFPEGKSFIADVEPIFDRLLFFWSDRRNPHEVQPSYATRYAMTVWYFDAEERAEAKKKFRNLTRKTESALTED

SEQ ID NO: 7 - DNA, 22 bp, synthetic construct
  misc_feature 1..22: note = Primer
  source 1..22: mol_type = other DNA
actcaacacgggaaacctcacc

SEQ ID NO: 8 - DNA, 20 bp, synthetic construct
  misc_feature 1..20: note = Primer
  source 1..20: mol_type = other DNA
ccagacaaatcgctccacca

SEQ ID NO: 9 - DNA, 20 bp, synthetic construct
  misc_feature 1..20: note = Primer
  source 1..20: mol_type = other DNA
caccatgccccacctacaat

SEQ ID NO: 10 - DNA, 20 bp, synthetic construct
  misc_feature 1..20: note = Primer
  source 1..20: mol_type = other DNA
tgggggtttttggccattct

SEQ ID NO: 11 - DNA, 21 bp, synthetic construct
  misc_feature 1..21: note = Primer
  source 1..21: mol_type = other DNA
aggagttcaatcacttggcgt

SEQ ID NO: 12 - DNA, 20 bp, synthetic construct
  misc_feature 1..20: note = Primer
  source 1..20: mol_type = other DNA
ccctccagcagttgagtagg

SEQ ID NO: 13 - DNA, 21 bp, synthetic construct
  misc_feature 1..21: note = Primer
  source 1..21: mol_type = other DNA
cctgcagtttggctacaacac

SEQ ID NO: 14 - DNA, 20 bp, synthetic construct
  misc_feature 1..20: note = Primer
  source 1..20: mol_type = other DNA
caggatgctctccccatagc

SEQ ID NO: 15 - DNA, 20 bp, synthetic construct
  misc_feature 1..20: note = Primer
  source 1..20: mol_type = other DNA
attacagcgatggggacaca

SEQ ID NO: 16 - DNA, 20 bp, synthetic construct
  misc_feature 1..20: note = Primer
  source 1..20: mol_type = other DNA
gccaaattggaaagagccga

SEQ ID NO: 17 - DNA, 20 bp, synthetic construct
  misc_feature 1..20: note = Primer
  source 1..20: mol_type = other DNA
gatcgtaggaacccacacga

SEQ ID NO: 18 - DNA, 20 bp, synthetic construct
  misc_feature 1..20: note = Primer
  source 1..20: mol_type = other DNA
tcagagcacggtcagtcttc

SEQ ID NO: 19 - DNA, 20 bp, synthetic construct
  misc_feature 1..20: note = Primer
  source 1..20: mol_type = other DNA
gcaaaatcaccaggacagcc

SEQ ID NO: 20 - DNA, 20 bp, synthetic construct
  misc_feature 1..20: note = Primer
  source 1..20: mol_type = other DNA
tctgttggcatggtgttcca

SEQ ID NO: 21 - DNA, 21 bp, synthetic construct
  misc_feature 1..21: note = Primer
  source 1..21: mol_type = other DNA
cggagcatcatccaggtctac

SEQ ID NO: 22 - DNA, 20 bp, synthetic construct
  misc_feature 1..20: note = Primer
  source 1..20: mol_type = other DNA
ggctggaagttgagtgccaa

(19) United States
(12) Patent Application Publication
(10) Pub. No.: US 2025/0250635 A1
(43) Pub. Date: Aug. 7, 2025

(54) KIT AND METHOD FOR DETERMINING PRESENCE OR ABSENCE OF AFFECTION OF DEMENTIA

(71) Applicants: KANEKA CORPORATION, Osaka (JP); Foundation for Biomedical Research and Innovation at Kobe, Hyogo (JP)

(72) Inventors: Akihiko Taguchi, Hyogo (JP); Yuka Okinaka, Hyogo (JP); Orie Saino, Hyogo (JP); Jun Tomono, Tokyo (JP); Yoshiyuki Shinagawa, Osaka (JP)

(73) Assignees: KANEKA CORPORATION, Osaka (JP); Foundation for Biomedical Research and Innovation at Kobe, Hyogo (JP)

(21) Appl. No.: 19/184,208

(22) Filed: Apr. 21, 2025

Related U.S. Application Data
(63) Continuation of application No. PCT/JP2023/038014, filed on Oct. 20, 2023.

(30) Foreign Application Priority Data
Oct. 21, 2022 (JP) 2022-169057

(51) Int. Cl. C12Q 1/6883

(52) U.S. Cl. CPC C12Q 1/6883; C12Q 2600/158

(57) ABSTRACT

Provided is a method, a kit, and a device for determining whether there is incidence of dementia or whether there is a risk of onset of dementia in a human subject using a biomarker that allows highly sensitive and specific determination by a simple operation. The method includes the step of calculating the ratio(s) between the expression level(s) of the connexin 43 gene and/or the prolyl hydroxylase domain-containing protein 3 gene, and the expression level of the connexin 37 gene.

### SEQUENCE LISTING

This application contains a sequence listing in computer readable form (File name: B220503 Sequence.xml; date of creation: Sep. 29, 2023; File size: 34 kb) which is incorporated herein by reference in its entirety and forms part of the disclosure.

### TECHNICAL FIELD

One or more embodiments of the present invention relates to a biomarker for determining whether there is incidence of dementia, a method for determining whether there is incidence of dementia or whether there is a risk of onset of dementia based on the biomarker, and a kit and a device.

### BACKGROUND

Due to the aging of the population, the number of cases of dementia is increasing, and attempts have been made to treat dementia by early detection and improvement of the lifestyle.

Amyloid PET (positron emission tomography) and tau PET are known as imaging methods for diagnosing dementia. However, these imaging methods are not suitable for the early determination of dementia due to a high cost, an insufficient number of facilities, a long time to be spent for testing, and others.

Further, cognitive function tests for determining dementia, such as the mini mental state examination (MMSE) and the Hasegawa dementia scale, are known. However, these cognitive function tests are not applicable to the determination of dementia in the early stage of its onset since the tests are to be used after the patients themselves have recognized the progression of the symptoms. Another problem with the cognitive function tests is that their results are ambiguous.

Another known method for determining dementia uses an examination of protein in cerebrospinal fluid, but this method is highly invasive, and not suitable for early diagnosis of dementia.

Examples of patent literature disclosing a method for determining whether there is incidence of dementia based on the gene expression level of a particular protein (biomarker) include Patent Literature 1 to 5.

Patent Literature 1 describes a method for testing Alzheimer's-type dementia, the method includes in vitro detection of a decrease in the level or function of at least 1 factor in the insulin/IGF signaling pathway, such as insulin, in a central nervous system tissue derived from a subject.

Patent Literature 2 describes a method in which phosphorylation of at least 1 substrate protein such as MARCKS in a subject is detected, and in which, when the degree of the phosphorylation is higher than in a normal sample, the subject is determined to be suffering from incidence of, or to have a risk of onset of, Alzheimer's-type dementia.

Patent Literature 3 describes a biomarker for detecting cognitive dysfunction disease. The biomarker described in Patent Literature 3 is described as a protein that is dependent on complement C4, prothrombin, complement C3, gelsolin, and the like.

Patent Literature 4 describes that the expression levels of genes related to metabolisms such as the respiratory chain, for example, the prolyl hydroxylase domain-containing protein 3 gene, are useful as biomarkers for dementia and the like.

Patent Literature 5 describes a method for screening an agent useful for the treatment of dementia and the like, using a biomarker described in Patent Literature 4.

### PATENT LITERATURE

Patent Literature 1: JP Patent Publication No. 2008-522199
Patent Literature 2: WO 2015/099094
Patent Literature 3: WO 2019/012671
Patent Literature 4: WO 2021/177447
Patent Literature 5: WO 2021/177448

### SUMMARY

As described above, biomarkers for dementia have been widely studied. However, a biomarker useful for definitive diagnosis often requires a complex operation for its detection. On the other hand, a biomarker detectable by a simple operation, including the prolyl hydroxylase domain-containing protein 3 gene, often has insufficient sensitivity and specificity for being used alone for definitive diagnosis.

Therefore, an object of one or more embodiments of the present invention is to provide a biomarker that allows the determination of whether there is incidence of dementia with high sensitivity and specificity by a simple operation, and to provide a method, a kit, and a device for the determination.

As a result of intensive study, the present inventors completed one or more embodiments of the following invention.

[1] A method for determining whether there is incidence of dementia or whether there is a risk of onset of dementia in a subject, wherein the method comprises the steps of: measuring the expression levels of the connexin 37 gene, and the connexin 43 gene and/or the prolyl hydroxylase domain-containing protein 3 gene in a sample derived from the subject; and calculating the ratio(s) between the expression level(s) of the connexin 43 gene and/or the prolyl hydroxylase domain-containing protein 3 gene, and the expression level of the connexin 37 gene, and wherein the subject is shown to be suffering from incidence of dementia or to have a risk of onset of dementia based on comparison of each ratio with a pre-determined cutoff value, or with a control value calculated in the same manner for a sample derived from a healthy individual.
[2] The method according to [1], wherein the sample is blood.
[3] The method according to [1] or [2], wherein the dementia is Alzheimer's-type dementia.
[4] A kit for determining whether there is incidence of dementia or whether there is a risk of onset of dementia, wherein the kit comprises: a reagent for measuring the expression level of the connexin 37 gene in a sample derived from a subject; and a reagent(s) for measuring the expression level(s) of the connexin 43 gene and/or the prolyl hydroxylase domain-containing protein 3 gene in the sample, and wherein each reagent comprises: a primer capable of amplifying all or part of the base sequence of the gene or the complementary base sequence thereto; and/or a probe capable of hybridizing with all or part of the base sequence of the gene or the complementary base sequence thereto.
[5] The kit according to [4], wherein each of the primer and the probe is a polynucleotide or a fragment thereof shown in any of the following (1) to (6): (1) a fragment consisting of 15 or more consecutive nucleotides selected from a polynucleotide consisting of the base sequence according to SEQ ID NO:1, 3 or 5; (2) a polynucleotide fragment having deletion, substitution, addition, or insertion of 1 or 2 bases in the base sequence of the polynucleotide fragment of (1); (3) a fragment comprising 15 or more consecutive nucleotides selected from a polynucleotide consisting of the base sequence according to SEQ ID NO:1, 3 or 5; (4) a polynucleotide fragment having deletion, substitution, addition, or insertion of 1 or 2 bases in the base sequence of the polynucleotide fragment of (3); (5) a polynucleotide that hybridizes under highly stringent conditions with a fragment sequence comprising 15 or more consecutive nucleotides selected from the base sequence according to SEQ ID NO:1, 3, or 5; and (6) a polynucleotide consisting of the base sequence complementary to the base sequence of the polynucleotide or a fragment thereof of (1) to (5).
[6] A kit for determining whether there is incidence of dementia or whether there is a risk of onset of dementia, wherein the kit comprises: a binding molecule for connexin 37 in a sample derived from a subject; and a binding molecule for connexin 43 and/or a binding molecule for prolyl hydroxylase domain-containing protein 3 in the sample, and wherein each binding molecule comprises an antibody or an active fragment thereof, or an aptamer, capable of binding to the target protein.
[7] A device for determining whether there is incidence of dementia or whether there is a risk of onset of dementia, wherein the device comprises: a reagent for measuring the expression level of the connexin 37 gene in a sample derived from a subject; and a reagent(s) for measuring the expression level(s) of the connexin 43 gene and/or the prolyl hydroxylase domain-containing protein 3 gene in the sample, and wherein each reagent comprises: a primer capable of amplifying all or part of the base sequence of the gene or the complementary base sequence thereto; and/or a probe capable of hybridizing with all or part of the base sequence of the gene or the complementary base sequence thereto.
[8] The device according to [7], wherein each of the primer and the probe is a polynucleotide or a fragment thereof shown in any of the following (1) to (6): (1) a fragment consisting of 15 or more consecutive nucleotides selected from a polynucleotide consisting of the base sequence according to SEQ ID NO:1, 3 or 5; (2) a polynucleotide fragment having deletion, substitution, addition, or insertion of 1 or 2 bases in the base sequence of the polynucleotide fragment of (1); (3) a fragment comprising 15 or more consecutive nucleotides selected from a polynucleotide consisting of the base sequence according to SEQ ID NO:1, 3 or 5; (4) a polynucleotide fragment having deletion, substitution, addition, or insertion of 1 or 2 bases in the base sequence of the polynucleotide fragment of (3); (5) a polynucleotide that hybridizes under highly stringent conditions with a fragment sequence comprising 15 or more consecutive nucleotides selected from the base sequence according to SEQ ID NO:1, 3, or 5; and (6) a polynucleotide consisting of the base sequence complementary to the base sequence of the polynucleotide or a fragment thereof of (1) to (5).
[9] A device for determining whether there is incidence of dementia or whether there is a risk of onset of dementia, wherein the device comprises: a binding molecule for connexin 37 in a sample derived from a subject; and a binding molecule for connexin 43 and/or a binding molecule for prolyl hydroxylase domain-containing protein 3 in the sample, and wherein each binding molecule comprises an antibody or an active fragment thereof, or an aptamer, capable of binding to the target protein.
[10] A biomarker for determining whether there is incidence of dementia or whether there is a risk of onset of dementia, wherein the biomarker is any one selected from the group consisting of the following (a) to (d): (a) polynucleotides consisting of the connexin 37 gene or a fragment thereof, and the connexin 43 gene or a fragment thereof; (b) polynucleotides consisting of the connexin 37 gene or a fragment thereof, and the prolyl hydroxylase domain-containing protein 3 gene or a fragment thereof; (c) polypeptides consisting of connexin 37 or a fragment thereof, and connexin 43 or a fragment thereof; and (d) polypeptides consisting of connexin 37 or a fragment thereof, and prolyl hydroxylase domain-containing protein 3 or a fragment thereof.

The present specification encompasses the contents disclosed in Japanese Patent Application No. 2022-169057, on which the priority of the present application is based.

According to the biomarker of one or more embodiments of the present invention, a biomarker that allows the determination of whether there is incidence of dementia with high sensitivity and specificity can be provided.

According to the determination method of one or more embodiments of the present invention, whether there is incidence of dementia can be determined with a simple method.

According to the kit and the device of one or more embodiments of the present invention, the biomarker can be simply measured.

### BRIEF DESCRIPTION OF THE DRAWINGS

FIG. 1 is a diagram showing the proportions of mononuclear cells in samples derived from healthy persons and dementia patients.

FIG. 2 is a diagram showing the expression levels of the connexin gene in mononuclear cell-comprising samples derived from healthy persons and dementia patients.

FIG. 3 is a diagram showing the expression levels of the connexin gene in whole-blood samples derived from healthy persons and dementia patients.

FIG. 4 is a diagram showing the relative value of the expression level of the connexin 37 gene to the expression level of each gene in mononuclear cell-comprising samples derived from healthy persons and dementia patients. In the figure, * represents p<0.05.

FIG. 5 is a diagram showing relative values of the expression level of the connexin 37 gene to the expression level of the prolyl hydroxylase domain-containing protein 3 (PHD3) gene in whole-blood samples derived from healthy persons and dementia patients. In the figure, * represents p<0.05.

### DETAILED DESCRIPTION

### 1. Biomarker

### 1-1. Overview

A first aspect of one or more embodiments of the present invention is a biomarker for determining whether there is incidence of dementia or whether there is a risk of onset of dementia. As a biomarker for determining whether there is incidence of dementia or whether there is a risk of onset of dementia in a subject, the biomarker of one or more embodiments of the present invention may be used in the determination method according to the second aspect.

### 1-2. Definitions

Terms used in the present specification are defined below.

“Dementia” refers to a disease whose core symptoms include memory impairment, disorientation, behavioral disorder, impaired comprehension and judgment, and executive function impairment that appear due to damage of brain cells. Dementia includes Alzheimer's-type dementia, vascular dementia, dementia with Lewy bodies, and frontotemporal dementia depending on the causative disease or disorder. Dementia in one or more embodiments of the present invention includes any of these, and its causative disease or disorder is not particularly limited.

“Alzheimer's-type dementia” refers to dementia that exhibits atrophy of the medial temporal lobe (hippocampus) in CT or MRI, and that involves event memory impairment as a core symptom of cognitive memory impairment. Alzheimer's-type dementia may involve visuospatial cognitive impairment such as constructional impairment or topographical disorientation; recent-memory impairment; and other kinds of cognitive impairment. Alzheimer's-type dementia is a general term for dementia caused by Alzheimer's disease, which is characterized by the aggregation of amyloid-β, and dementia that exhibits symptoms similar to those of Alzheimer's disease.

“Vascular dementia” refers to dementia that appears in association with a cerebrovascular disorder, and that involves impairment in complex attentional abilities such as information-handling speed, and in the frontal lobe-mediated executive function. Depending on the type of vascular disorder, This type of dementia is classified into multiple infarct dementia, small vessel disease with dementia, hypoperfusion-type vascular dementia, hemorrhagic vascular dementia, and the like. Vascular dementia is frequently comorbid with Alzheimer's-type dementia.

“Dementia with Lewy bodies” refers to dementia that shows, as a main lesion, the appearance of inclusion bodies (Lewy bodies) due to abnormal accumulation of α-synuclein in neurons, and that involves symptoms such as cognitive fluctuation, visual hallucination, and/or idiopathic parkinsonism. Dementia with Lewy bodies may also involve constructional impairment, severe hypersensitivity to antipsychotics, REM sleep behavior disorder, and/or the like. Lewy bodies may also be found outside the central nervous system.

“Frontotemporal dementia” refers to dementia that exhibits brain atrophy in the frontal and/or temporal lobes, and that involves progressive aphasia. Further, although frontotemporal dementia may involve, for example, social cognitive impairments such as personality change, and executive dysfunction, memory impairment is often relatively mild. Frontotemporal dementia can be roughly divided into pathological conditions showing tau accumulation and pathological conditions showing TDP-43 accumulation. In addition, pathological conditions showing FUS (fused in sarcoma) are also known.

The term “subject” herein refers to a human or non-human animal to be subjected to the determination of whether there is incidence of dementia or whether there is a risk of onset of dementia. For example, a non-human animal includes non-human mammals such as primate animals, rats, mice, Mongolian gerbils, guinea pigs, hamsters, ferrets, rabbits, cows, horses, pigs, goats, dogs, and cats. For example, the subject includes an individual suffering from dementia, an individual with a possibility of future onset of dementia, or a healthy individual.

The term “determining whether there is incidence” herein refers to judgment of whether a patient is suffering from, or not suffering from, a particular disease. This includes differential identification from other diseases that exhibit similar lesions or symptoms.

The term “risk of onset” herein refers to the degree of risk at which transition from the current state to a particular disease state in the future, resulting in suffering from the particular disease and the appearance of its symptoms. Specifically, for example, the method of representing the risk of onset includes representation by a probability assuming that the probability is 100% when the onset inevitably occurs, representation by the stage, or representation by an approximate period before the onset. Further, “there is a risk of onset” refers to the fact that the degree of risk is at a certain level or higher.

The term “individual with a possibility of future onset of dementia” herein refers to an individual with a high risk of onset of dementia. Specifically, for example, the individual includes an individual who exhibits symptoms found in dementia, such as cognitive decline; an individual who has once been determined to have a risk of onset of dementia based on information such as a biomarker; or an individual having a risk factor of dementia such as advanced age, hearing loss, smoking, depression, diabetes, or hypertension.

“Healthy individual” refers to an individual in a healthy state. In the present description, “healthy state” refers to a state where an individual is at least not suffering from the disease to be compared. Therefore, an individual who is at least not suffering from dementia is considered to be a healthy individual even when the individual is suffering from other diseases. The healthy individual is particularly an individual who is healthy and shows no incidence of any disease. In the present specification, the healthy individual is used mainly as a control individual that serves as a basis for comparison with a subject. Therefore, the healthy individual herein is of the same species as the subject. Further, the healthy individual particularly has the same conditions in terms of, for example, the subspecies (such as the race), the age (such as the age in month or week), the sex, the body height, and the body weight.

“Sample derived from a subject” refers to a tissue, a cell, or a body fluid that is collected from a subject, and that is used in each aspect of one or more embodiments of the present invention. For example, samples that may be used herein include body fluid samples such as blood (including peripheral blood), cerebrospinal fluid, saliva, urine, sputum, sweat, pharyngeal swab, and nasal swab; central nervous system tissues such as bone marrow and brain tissues; vascular tissues; skin tissues; or fat tissues. For example, the blood includes those in the forms of serum, plasma, or whole blood. For example, the bone marrow-derived sample includes bone marrow fluid. Further, as a body fluid sample or a bone marrow sample derived from the subject, for example, a sample containing a cell having 1 nucleus, isolated from a body fluid or bone marrow of the subject may be used.

“Leukocyte” is a general term for nucleated cells among the cells contained in blood. Red blood cells and platelets are not included in white blood cells. Leukocytes include polymorphonuclear cells and mononuclear cells. Polymorphonuclear cells include eosinophils, neutrophils, and basophils, and are characterized by the presence of polymorphonuclei and many cytoplasmic granules. For this reason, polymorphonuclear cells are also called granulocytes.

Among leukocytes, “peripheral blood mononuclear cells (PBMC)” refers to cells having a single round nucleus. Mononuclear cells include, for example, lymphocytes and monocytes. Although the term “mononuclear cells” usually refers to only those cells present in peripheral blood, the “mononuclear cells” in the present specification broadly include lymphocytes and monocytes, including mononuclear cells that have moved to other sites by migration or the like.

The term “cutoff value” herein refers to a value that can be used as a standard for the determination of whether there is incidence of a disease or whether there is a risk of onset of a disease. In general, a cutoff value is derived by using a known method from an ROC curve drawn based on a direct comparison between a control group for comparison and a disease group. However, the method for determination of the cutoff value is not particularly limited thereto. For example, a cutoff value may be set by a method without using a direct comparison between a control group for comparison and a disease group, or a method without using an ROC curve. Any known method may be used as the method for determination of the cutoff value using an ROC curve. Specifically, for example, the method includes a method using the Youden index or a method using the distance from the upper left corner (the point where both the sensitivity and the specificity are 100%).

“ROC curve (Receiver Operating Characteristic curve)” is prepared by plotting the True Position Fraction (TPF), that is, the sensitivity, on the ordinate, and the False Position Fraction (FPF), that is, (1—specificity), on the abscissa, while changing the threshold to determine which values in the determination result are regarded as being abnormal, in other words, the cutoff point, as a parameter. Here, the sensitivity is the rate at which positive persons are accurately judged as positive, and the specificity is the rate at which negative persons are accurately judged as negative.

“AUC (Area Under the Curve)” is the value of the area under the ROC curve, and is a numerical value that represents the judgment ability of an index associated with the ROC curve. In general, an AUC closer to 1 is considered to indicate a higher judgment ability, and an AUC closer to 0.5 is considered to indicate a lower judgment ability.

The term “significance” herein refers to statistical significance. The statistical significance refers to the presence of a significant difference between a measured value of a test subject and a control value in statistical processing of their difference. For example, an obtained value is judged to be significant when the level of significance (significance level) of the obtained value is small, specifically, when it is smaller than 5% (p<0.05), when it is smaller than 1% (p<0.01), or when it is smaller than 0.1% (p<0.001). The “p (value)” shown herein indicates the probability that a test statistic agrees with the value by chance within a distribution based on the null hypothesis in a statistical test. Therefore, the smaller the “p”, the lower the probability that the test statistic will be that value, indicating that the null hypothesis is more likely to be rejected. The test method for the statistical processing is not particularly limited, and any known test method capable of judging the presence or absence of significance may be used as appropriate. For example, the Student's t-test method, the paired Student's t-test method, the Welch's t-test method, the Wilcoxon rank-sum test, analysis of variance, the Tukey post hoc test, or the like may be used, but is not particularly limited thereto.

The term “biomarker” herein refers to a biomolecule (for example, a nucleic acid molecule or a protein molecule) that serves as an index for determining whether there is incidence of a disease or whether there is a risk of onset of a disease.

“One or plural” refers to 1 to 10, particularly 1 to 5, more particularly 1 to 4, more particularly 1 to 3, especially particularly 1 or 2.

### 1-3. Constitution

The biomarker of one or more embodiments of the present invention is composed of a combination of two or more kinds of proteins or genes, or fragments thereof.

### 1-3-1. Factors Constituting Biomarker

The biomarker of one or more embodiments of the present invention consists of a combination of the genes or proteins, or fragments thereof, of connexin 37, and connexin 43 or prolyl hydroxylase domain-containing protein 3. Each factor is described below.

### (1) Connexin 37

“Connexin 37 (Cx37)” is a protein belonging to the connexin family and is a cell membrane protein with a molecular weight of about 37 kDa constituting the gap junction. Further, the “connexin 37 gene (Cx37 gene)” refers to the gene encoding connexin 37. Connexin 37 is also known by other names such as GJA4, and mutations in this gene have been reported to be associated with an increased risk of arteriosclerosis and myocardial infarction.

The specific amino acid sequence of the connexin 37 and the base sequence of its gene are not particularly limited. The amino acid sequence of the protein is, for example, the amino acid sequence of human connexin 37 (333 amino acids) according to SEQ ID NO:2. Further, the base sequence of the gene is, for example, the base sequence according to SEQ ID NO:1 (1621 bases), which encodes the human connexin 37 according to SEQ ID NO:2.

### (2) Connexin 43

“Connexin 43 (Cx43)” is a protein belonging to the connexin family and is a cell membrane protein with a molecular weight of about 43 kDa constituting the gap junction. Further, the “connexin 43 gene (Cx43 gene)” refers to the gene encoding connexin 43. Connexin 37, also known by other names such as GJA1, has been thought to play an important role, particularly in synchronized contraction of cardiac muscle. Mutations in this gene have been reported to be involved in dysplasia of the eyes/teeth/digits, skull, and heart.

The specific amino acid sequence of the connexin 43 and the base sequence of its gene are not particularly limited. The amino acid sequence of the protein is, for example, the amino acid sequence of human connexin 43 (382 amino acids) according to SEQ ID NO:4. Further, the base sequence of the gene is, for example, the base sequence according to SEQ ID NO:3 (3083 bases), which encodes the human connexin 43 according to SEQ ID NO:4.

### (3) Prolyl Hydroxylase Domain-Containing Protein 3

“Prolyl hydroxylase domain-containing protein 3 (PHD3)” is a protein belonging to the PHD (prolyl hydroxylase domain-containing protein) family. Further, the “prolyl hydroxylase domain-containing protein 3 gene (PHD3 gene)” refers to the gene encoding PHD3. PHD3, also known by names such as EGLN3 and HPH1, is a protein present in the cytoplasm or nucleus, and is known as a protein involved in apoptosis and hypoxic response.

The specific amino acid sequence of the PHD3 and the base sequence of its gene are not particularly limited. The amino acid sequence of the protein is, for example, the amino acid sequence of human PHD3 (239 amino acids) according to SEQ ID NO:6. Further, the base sequence of the gene is, for example, the base sequence of the human PHD3 gene according to SEQ ID NO:5 (2706 bases), which encodes the human prolyl hydroxylase domain-containing protein 3 according to SEQ ID NO:6.

Each of the base sequences according to SEQ ID NOs: 1, 3, and 5 includes a base sequence encoding the amino acid sequence of the protein (CDS), and the base sequences of the untranslated regions (UTRs) located upstream and downstream of the CDS.

### 1-3-2. Constitution of Biomarker

The biomarker of one or more embodiments of the present invention consists of the following combinations of the above-described 3 factors.
(a) Polynucleotides consisting of the connexin 37 gene or a fragment thereof, and the connexin 43 gene or a fragment thereof (often referred to as “Cx37 gene or the like/Cx43 gene or the like” herein);
(b) polynucleotides consisting of the connexin 37 gene or a fragment thereof, and the prolyl hydroxylase domain-containing protein 3 gene or a fragment thereof (often referred to as “Cx37 gene or the like/PHD3 gene or the like” herein);
(c) polypeptides consisting of connexin 37 or a fragment thereof, and connexin 43 or a fragment thereof (often referred to as “Cx37 or the like/Cx43 or the like” herein); and
(d) polypeptides consisting of connexin 37 or a fragment thereof; and prolyl hydroxylase domain-containing protein 3 or a fragment thereof (often referred to as “Cx37 or the like/PHD3 or the like” herein).

The fragment in the biomarker of one or more embodiments of the present invention is not particularly limited as long as the presence of the fragment indicates the expression of the target protein or gene rather than the expression of other proteins or genes. The length of the fragment is not particularly limited. Specifically, when the fragment is a polynucleotide, it has, for example, 15 bases or more, 17 bases or more, 19 bases or more, 20 bases or more, 25 bases or more, 30 bases or more, 40 bases or more, 50 bases or more, 100 bases or more, 200 bases or more, 300 bases or more, 500 bases or more, 1000 bases or more, 1500 bases or more, or 2000 bases or more. Further, when the fragment is a polypeptide, it has, for example, 10 amino acids or more, 20 amino acids or more, 30 amino acids or more, 40 amino acids or more, 50 amino acids or more, 100 amino acids or more, 150 amino acids or more, 200 amino acids or more, or 300 amino acids or more.

### 2. Determination Method

### 2-1. Overview

A second aspect of one or more embodiments of the present invention is a method for determining whether there is incidence of dementia or whether there is a risk of onset of dementia. By the method of the present aspect, whether there is incidence of dementia or whether there is a risk of onset of dementia in a subject can be determined based on the biomarker according to the first aspect.

### 2-2. Method

The determination method of one or more embodiments of the present invention comprises the steps of measuring the amount of a biomarker and calculating the ratio of the expression level as essential steps, and whether there is incidence of dementia or whether there is a risk of onset of dementia is indicated based on the calculated ratio. Each step is specifically described below.

### 2-2-1. Step of Measuring Amount of Biomarker

The present step is a step of measuring the amount of the biomarker according to the first aspect, in a sample derived from a subject.

When a biomarker consisting of polynucleotides is used, the present step is a step of measuring the expression levels of the Cx37 gene, and the Cx43 gene and/or the PHD3 gene in a sample derived from a subject.

On the other hand, when a biomarker consisting of polypeptides is used, the present step is a step of measuring the amounts of Cx37, and Cx43 and/or PHD3 proteins in a sample derived from a subject.

The present step uses a predetermined amount of sample derived from the subject.

The biological species of the subject in the method of the present aspect is not particularly limited. For example, the subject may be any mammal as described in the Definitions section. The subject is particularly a non-human primate animal or human.

The conditions of the subject in the method of the present aspect are not particularly limited. For example, the subject may be any of an individual suffering from dementia, an individual with a possibility of future onset of dementia, an individual suspected of having dementia, or an apparently healthy individual.

For example, the sample used in the present step includes a body fluid, bone marrow, or central nervous system tissue. From the viewpoint of invasiveness upon the sample collection, the sample is particularly blood, saliva, urine, sputum, sweat, pharyngeal swab, nasal swab, or the like. Basically, when a control value is used later for comparison, the samples of the subject and the healthy individual for obtaining the control value should be of the same type. For example, when one of them is blood, the other should also be blood.

“Predetermined amount” refers to an amount preliminarily determined in terms of volume or weight. Although the predetermined amount is not particularly limited, it at least needs to be an amount by which the biomarker according to the first aspect contained in the sample can be measured. For example, when blood is used as the sample, the predetermined amount may be, for example, 100 μL to 20 mL.

The method for collecting the sample may be a known method and is not particularly limited. For example, when the sample is a bone marrow fluid, it may be collected by lliac puncture. In cases where the sample is blood, saliva, urine, sputum, sweat, pharyngeal swab, or nasal swab, the sample may be collected according to a known method.

The sample used in one or more embodiments of the present invention is particularly a sample comprising a cell having a nucleus. The type of the cell contained in the sample is not particularly limited. For example, when the sample is blood, the cell is particularly a mononuclear cell.

For example, when the sample comprises mononuclear cells, the ratio of the mononuclear cells in the sample is not particularly limited. The ratio may be, for example, 10% or more, 20% or more, 30% or more, 40% or more, 50% or more, 60% or more, 70% or more, 80% or more, 90% or more, 95% or more, 98% or more, 99% or more, or 100%. The term “ratio of mononuclear cells in the sample” herein refers to the proportion of the number of mononuclear cells to the total number of leukocytes contained in the sample. The ratio of mononuclear cells in the sample may be, but does not necessarily need to be, measured by, for example, a blood cell analyzer, a hematocytometer, flow cytometry, or the like.

Further, as the sample, a sample comprising cells isolated from the subject may also be used. The cells may be nonspecifically isolated regardless of their type, or particular cells may be specifically isolated. When the sample comprises cells, the cells particularly comprise mononuclear cells. In the present specification, a sample comprising isolated mononuclear cells is specifically referred to as a mononuclear cell-comprising sample.

The ratio of mononuclear cells in the mononuclear cell-comprising sample is not particularly limited. The ratio may be, for example, 50% or more, 60% or more, 70% or more, 80% or more, 90% or more, 95% or more, 98% or more, 99% or more, or 100%. Further, the ratio of granulocytes in the mononuclear cell-comprising sample is particularly less than 50%, less than 40%, less than 30%, less than 20%, less than 10%, less than 5%, less than 2%, or 0%. The term “ratio of mononuclear cells or granulocytes in the mononuclear cell-comprising sample” herein refers to the proportion of the number of mononuclear cells or granulocytes to the total number of cells contained in the mononuclear cell-comprising sample. The ratio of mononuclear cells or granulocytes in the mononuclear cell-comprising sample may be, but does not necessarily need to be, measured by, for example, a blood cell analyzer, a hematocytometer, flow cytometry, or the like.

A sample derived from a subject may comprise granulocytes, and the granulocytes in the sample may be a factor that decreases the measurement accuracy of the amount of the biomarker. Thus, the mononuclear cell-comprising sample used may be a sample obtained by increasing the ratio of mononuclear cells by separating mononuclear cells or removing granulocytes from the sample. By subjecting the mononuclear cell-comprising sample to the measurement, a highly accurate measured value can be obtained.

The method for obtaining the mononuclear cell-comprising sample from a body fluid, especially blood or bone marrow, derived from the subject is particularly a method using a reagent and/or a column for separating mononuclear cells. For example, the reagent and/or the column include Ficoll-Paque PLUS (manufactured by GE Healthcare), Lymphoprep (manufactured by Abbott Diagnostics Technologies), Human Peripheral Blood Mononuclear Cell Isolation and Viability Kit (manufactured by BioVision), BD Vacutainer® CPT blood collection tube for separation of mononuclear cells (manufactured by Becton Dickinson), pluriMate (manufactured by pluriSelect), and SepMate (manufactured by STEMCELL Technologies). By using the reagent and/or the column, mononuclear cells and granulocytes in the sample can be separated from each other to allow efficient acquisition of the mononuclear cell-comprising sample to be subjected to the measurement.

In the present step, the amount of the biomarker according to the first aspect in the above-described sample is measured. The term “amount of the biomarker” herein is a value indicating the amount of each factor in the biomarker measured in the present step. When the biomarker consists of polynucleotides, the “amount of the biomarker” is the “expression levels”, that is, the “amounts of transcription products (including the amounts of cDNAs)”. On the other hand, when the biomarker consists of polypeptides, the “amount of the biomarker” is the “amount of proteins”.

The amount of the biomarker may be an absolute value such as volume or weight, or may be a relative value such as the concentration, ionic strength, absorbance, or fluorescence intensity. Further, the amount of the biomarker may be a value standardized by the amount of an endogenous control such as a housekeeping gene, or a protein that is a transcription product thereof. However, in the method of one or more embodiments of the present invention, standardization by endogenous control is not necessarily required since the quotient between the gene expression levels or the amounts of the proteins is calculated later in the step of calculating the relative value.

As the endogenous control, housekeeping genes represented by TBP (TATA-binding protein), GAPDH (glyceraldehyde-3-phosphate dehydrogenase), β-actin, β2M (2 microglobulin), HPRT1 (hypoxanthine phosphoribosyltransferase 1), and 18S ribosomal RNA may be used.

The amount of the biomarker may be measured by detecting the amounts of mRNA (including the coding region and the untranslated regions) of the target genes in the sample. Further, the amount of the biomarker may also be measured by detecting the protein amounts of the target proteins in the sample.

For the detection of the mRNA, for example, Northern blotting, the RT-PCR method, the real-time RT-PCR method, the nucleic acid array method (a method using a nucleic acid chip), the dot blotting method, the RNase protection assay method, or the in situ hybridization method may be used. These methods may be carried out by known procedures. Further, the measurement in the present step may be carried out using the primer and/or the probe according to a third aspect.

When Northern blotting is used, the presence or absence of the expression, and the expression level, of each gene in RNA can be detected and measured by using the probe according to the third aspect. Specifically, it may be carried out by the following procedure. First, a probe (complementary strand) is preliminarily labeled with a radioisotope (such as ³²P, ³³P, or ³⁵S), a fluorescent substance, or the like. Subsequently, RNA in a sample derived from a subject is transferred to a nylon membrane or the like. The labeled probe is applied to this membrane, to allow the labeled probe to hybridize with the RNA. Finally, a signal derived from the label in the DNA/RNA double strand formed is detected and measured by a radiation detector, fluorescence detector, or the like.

When the quantitative RT-PCR method is used, the presence or absence of gene expression and its expression level in RNA can be detected and measured by using the primer according to the third aspect. Specifically, it may be carried out by the following procedure. First, cDNA is prepared from RNA in a sample derived from a subject. Subsequently, using the cDNA as a template, the PCR method is carried out with a pair of primers capable of amplifying the target nucleic acid region, and the resulting double-stranded DNA is detected. Specifically, for example, the detection method for the double-stranded DNA includes a method in which the PCR described above is carried out using primers preliminarily labeled with a radioisotope or a fluorescent substance, a method in which the PCR product is subjected to agarose electrophoresis followed by staining of double-stranded DNA with ethidium bromide or the like, and a method in which the double-stranded DNA produced is transferred to a nylon membrane or the like according to a conventional method, and then detected by hybridization with a labeled probe.

When nucleic acid array analysis is utilized, an RNA or DNA chip having a solid phase such as a substrate on which the probe according to the third aspect is immobilized is used. In this method, nucleic acid molecules obtained from a sample derived from a subject are applied to the chip, and a signal from the chip is detected to detect the presence or absence of the expression of a target gene.

A substrate having nucleic acid immobilized thereon is generally known by the names of a nucleic acid chip, a nucleic acid array, a microarray, and the like. Further, DNA or RNA arrays also include DNA or RNA macroarrays, and DNA or RNA microarrays. The chip herein includes any of these.

The method for measuring the signal from the chip is not particularly limited. For example, the method includes a method in which a signal derived from a label in a detection composition is detected and measured by an image detector (for example, Typhoon 9410 (GE Healthcare) or a 3D-Gene® Scanner (Toray Industries, Inc.)).

“Surface Plasmon Resonance (SPR) method” is a method for highly sensitively detecting and quantifying an adsorbate on the surface of a metal thin film utilizing the surface plasmon resonance phenomenon. The surface plasmon resonance phenomenon is a phenomenon in which, when the incident angle of laser light irradiated on a metal thin film is changed, the reflected light intensity is significantly attenuated at a specific incident angle (resonance angle). In one or more embodiments of the present invention, a nucleic acid probe having a sequence complementary to the base sequence of the target nucleic acid molecule, which is a biomarker, is immobilized on a surface of a metal thin film, and the remaining surface area of the metal thin film is subjected to blocking treatment, followed by allowing a liquid sample such as a body fluid collected from a subject to flow on the metal thin film surface. As a result, base pairs are formed between the target nucleic acid molecule and the nucleic acid probe. Thereafter, based on the difference between the measured values before and after the flowing of the sample, the target nucleic acid molecule is detected and quantified. For the detection and quantification by the surface plasmon resonance method, for example, an SPR sensor, which is commercially available from Biacore, may be used. This technique is well known in the art. See, for example, Kazuhiro Nagata and Hiroshi Handa. Real-Time Analysis of Biomolecular Interactions (Springer-Verlag Tokyo, Tokyo, 2000).

The “Quartz Crystal Microbalance (QCM) method” is a mass measurement method that utilizes a phenomenon in which adsorption of a substance to the surface of an electrode attached to a quartz crystal results in a decrease in the resonance frequency of the quartz crystal, to quantitatively capture an extremely small amount of adsorbate based on the amount of change in the resonance frequency. For the detection and quantification by the present method, a commercially available QCM sensor may be used in the same manner as in the SPR method. For example, a target nucleic acid molecule can be detected and quantified based on base pairing between a nucleic acid probe immobilized on an electrode surface, having the sequence complementary to the base sequence of the target nucleic acid molecule, and the target nucleic acid molecule in a sample collected from a subject. This technique is well known in the art. See, for example, J. Christopher Love, L. A. Estroff, J. K. Kriebel, R. G. Nuzzo, G. M. Whitesides (2005) Self-Assembled Monolayers of a Form of Nanotechnology, Chemical Review, 105:1103-1170; Toyosaka Moriizumi and Takamichi Nakamoto (1997), Sensor Engineering, Shokodo Co., Ltd.

For the calculation of the gene expression level, for example, the statistical processing described in Statistical analysis of gene expression microarray data (Speed T. (author), Chapman and Hall/CRC) and A beginner's guide Microarray gene expression data analysis (Causton H. C. et al. (authors), Blackwell publishing) may be used in one or more embodiments of the present invention although the calculation is not particularly limited thereto. For example, 2, 3, or 6 times the standard deviation of measured values of blank spots on the DNA chip may be added to the average of the measured values of the blank spots, and probe spots showing signal values that are not less than the resulting value may be regarded as detected spots. Further, the average value of the measured values of the blank spots may be regarded as the background, and the value obtained by subtracting the background from the measured value of each probe spot may be regarded as the gene expression level.

Each missing value of the gene expression level may be excluded from the analysis, or may particularly be replaced by the minimum value of the gene expression level in each DNA chip, or may more particularly be replaced by the value calculated by subtracting 0.1 from the logarithmic value of the minimum value of the gene expression level. Further, for the removal of low-signal genes, only genes showing gene expression levels of 2⁶ or more, 2⁸ or more, or 2¹⁰ or more in 20% or more, 50% or more, or 80% or more of the number of measured samples may be selected as analysis targets.

The amount of protein may be detected by an immunoassay using an antibody that specifically recognizes and binds to the target protein. The antibody may be prepared by a known method. For example, the immunoassay includes a method using a solid-phase carrier on which an antibody that specifically binds to the target protein to be detected is immobilized, flow cytometry, and Western blotting. For example, the method using a solid-phase carrier includes an enzyme-linked immunosorbent assay (ELISA) using a microtiter plate for the immobilization and an agglutination method (immunoprecipitation method) using particles for the immobilization, but is not particularly limited thereto. Known immunological assays may be employed to detect the amount of the target protein in the sample. Further, the amount of the target protein may also be detected by a method using, for example, LC-MS/MS MRM, which is a protein mass spectrometry technique without using an antibody. These detection methods may also be carried out according to conventional protocols. Further, for the measurement in the present step, the peptide-binding molecule according to the third aspect may be used.

Before the measurement of the amount of the biomarker by the present step, an optional treatment may be carried out if necessary. For example, the treatment includes extraction of nucleic acid or protein from the sample.

The method for extracting the nucleic acid from the sample is not particularly limited. The nucleic acid may be extracted using a conventionally known buffer, apparatus, kit, or the like under conditions where the target polynucleotide is not degraded. For example, the nucleic acid may be extracted by the acid phenol method, the phenol/chloroform extraction method, or the like. As a method for collecting nucleic acid from the extraction solution, for example, the ethanol precipitation method may be used. Further, a commercially available nucleic acid extraction kit may also be used. As the nucleic acid extraction kit, the DNeasy Blood & Tissue Kit (QIAGEN), the Quick-DNA kit (Zymoresearch), or the like may be used. Further, a reagent or kit for specific extraction of RNA may also be used.

In the method of one or more embodiments of the present invention, the method for extracting nucleic acid from the sample may be, for example, the use of the 3D-Gene® RNA extraction reagent from liquid sample kit (Toray Industries, Inc.).

Further, the method for extracting protein from the sample is not particularly limited. The protein may be extracted using a conventionally known buffer, apparatus, kit, or the like under conditions where the target protein is not degraded. For example, the protein may be extracted by using a protein denaturant and/or by mechanical homogenization treatment. For example, the protein denaturant includes urea, guanidine salt, trichloroacetic acid (TCA), and surfactants. Further, for example, the surfactant includes SDS (sodium lauryl sulfate) CHAPS, Triton X-100, Tween 20, and Nonidet P-40. For example, the mechanical homogenization treatment includes homogenization using a homogenizer, ultrasonication, French press treatment, and homogenization using glass beads.

### 2-2-2. Step of Calculating Ratio

The present step is a step of calculating the ratio between the expression level of the Cx37 gene or the amount of protein of Cx37, and the expression level of another gene or the amount of another protein. The present step may be carried out at the same time as or after the step of measuring the amount of the biomarker.

When the measured biomarker consists of a polynucleotide, the ratio(s) between the expression level of the Cx43 gene and/or the expression level of the PHD3 gene, and the expression level of the Cx37 gene, is/are calculated in the present step.

When this ratio is calculated, the ratio is a relative value of the expression level of the Cx37 gene to the expression level of the Cx43 gene and/or the expression level of the PHD3 gene (hereinafter often simply referred to as “relative value of the expression level of the Cx37 gene” in the present specification), or the reciprocal thereof (a relative value of the expression level of the Cx43 gene and/or the expression level of the PHD3 gene to the expression level of the Cx37 gene (hereinafter often simply referred to as “relative value to the expression level of the Cx37 gene” in the present specification)).

When the measured biomarker consists of polypeptides, the ratio between the amount of protein of Cx43 and/or the amount of protein of PHD3, and the amount of protein of Cx37, is calculated in the present step.

When this ratio is calculated, the ratio is a relative value of the amount of protein of Cx37 to the amount of protein of Cx43 and/or the amount of protein of PHD3 (hereinafter often simply referred to as “relative value of the amount of protein of Cx37” in the present specification), or the reciprocal thereof (a relative value of the amount of protein of Cx43 and/or the amount of protein of PHD3 to the amount of protein of Cx37 (hereinafter often simply referred to as “relative value to the amount of protein of Cx37” in the present specification)).

The term “ratio” refers to the quotient value between values of a plurality of indexes.

The “relative value” of a first value to a second value refers to a value obtained as a result of comparing the first value with the second value, and usually refers to the value obtained by dividing the first value by the second value.

The means for calculating the relative value in the present step is not particularly limited. For example, the relative value may be manually calculated or may be calculated using a computing device.

The subject is shown to be suffering from dementia or to have a risk of onset of dementia based on a comparison of a pre-determined cutoff value, or a control value calculated in the same manner for a sample derived from a healthy individual, with the ratio calculated in the present step. This may be determined by a step of determination. The detail of determination is described in the step of determination.

### 2-2-3. Step of Determination

The present step is a step in which the subject is determined to be suffering from incidence of dementia or to have a risk of onset of dementia based on the ratio. The present step may be carried out at the same time as or after the step of calculating the ratio.

The determination may be based on a comparison of the calculated ratio with a cutoff value, or with a control value.

When a cutoff value is used in the present step, the subject is determined to be suffering from dementia or to have a risk of onset of dementia when the relative value of the expression level of the Cx37 gene or of the amount of protein of Cx37 is lower than a pre-determined cutoff value, or when the relative value to the expression level of the Cx37 gene or to the amount of protein of Cx37 is higher than a pre-determined cutoff value.

The specific value of the cutoff value may vary depending on the biomarker, the sample used, and the measurement method for the biomarker used in the method of one or more embodiments of the present invention. For example, the cutoff value for the relative value of the expression level of the Cx37 gene to the expression level of the PHD3 gene is 0.893 according to the result of Example 3 when the expression levels are measured by the detection of nucleic acid in a mononuclear cell-comprising sample, and the cutoff value is 0.744 according to the result of Example 4 when the expression levels are measured in the same manner in whole blood. Further, the cutoff value for the relative value of the expression level of the Cx37 gene to the expression level of the Cx43 gene is 0.651 according to the result of Example 3 when the expression levels are measured by the detection of nucleic acid in a mononuclear cell-comprising sample. Further, similarly, for example, the cutoff value for the relative value of the expression level of the PHD3 gene to the expression level of the Cx37 gene is 1.120 when the expression levels are measured by the detection of nucleic acid in a mononuclear cell-comprising sample, and the cutoff value is 1.344 when the expression levels are measured in the same manner in whole blood. Further, the cutoff value for the relative value of the expression level of the Cx37 gene to the expression level of the Cx43 gene is 1.536 when the expression levels are measured by the detection of nucleic acid in a mononuclear cell-comprising sample.

The cutoff value used in one or more embodiments of the present invention particularly achieves sufficiently high sensitivity and specificity. For example, both sensitivity and specificity of the cutoff value are particularly 65% or more, 70% or more, 75% or more, 80% or more, 85% or more, or 90% or more. Further, regarding the AUC, which indicates the judgment ability of the biomarker, the cutoff value particularly achieves an AUC of 0.75 or more, 0.78 or more, 0.79 or more, 0.8 or more, 0.85 or more, 0.88 or more, 0.89 or more, or 0.9 or more.

When a control value is used in the present step, the subject is usually determined to be suffering from incidence of dementia or to have a risk of onset of dementia when a relative value of the expression level of the Cx37 gene or of the amount of protein of Cx37 is significantly lower than the control value, or when a relative value to the expression level of the Cx37 gene or to the amount of protein of Cx37 is significantly higher than the control value.

In one or more embodiments of the present invention, “control value” refers to a relative value calculated for a sample derived from a healthy individual in the same manner as the subject. Therefore, the subject may be determined to be suffering from incidence of dementia or to have a risk of onset of dementia when the relative value is significantly lower or higher than a control value calculated in the same manner for a sample derived from a healthy individual.

The specific method for determining the control value is not particularly limited. For example, the average of relative values preliminarily measured and calculated for healthy individuals using the same method may be used as the control value, or the average of relative values obtained from healthy individuals at the same timing as the subject may be used as the control value. When a preliminarily calculated control value is used, a confidence interval corresponding to a desired significance level is particularly provided together with the control value. When such a confidence interval is provided, the subject may be determined to be suffering from incidence of dementia or to have a risk of onset of dementia when the relative value of the subject is lower or higher than the confidence interval.

When the control value is used, the significance level is not particularly limited. Specifically, for example, any of the significance levels described in the Definitions section may be used. Additionally, depending on the purpose and the like, any other commonly used significance level may be used. Further, depending on the purpose, a plurality of significance levels may be used in the determination.

Further, the subject may be determined to be suffering from incidence of dementia or to have a risk of onset of dementia when the relative value of the subject is lower or higher than the control value by a certain extent or more. When a relative value of the expression level of the Cx37 gene or of the amount of protein of Cx37 is used, the ratio of the relative value to the control value may be, for example, 0.9 or more, 0.8 or more, 0.7 or less, 0.6 or less, 0.5 or less, 0.4 or less, 0.3 or less, 0.2 or less, or 0.1 or less. Further, when a relative value to the expression level of the Cx37 gene or to the amount of protein of Cx37 is used, the ratio of the relative value to the control value may be, for example, not 1.1 or more, 1.2 or more, 1.3 or more, 1.4 or more, 1.5 or more, 1.6 or more, 1.7 or more, 1.8 or more, 1.9 or more, 2.0 or more, 3.0 or more, 5.0 or more, or 10 or more.

In the present aspect, a plurality of values may be used as each of the cutoff value and the control value. For example, the cutoff value or the control value for determining whether there is a risk of onset of dementia, and the cutoff value or the control value for determining whether there is incidence of dementia may be separately determined. When a relative value of the expression level of the Cx37 gene or of the amount of protein of Cx37 is used, the subject may be determined to have a risk of onset dementia when, for example, the relative value in the subject is lower than a cutoff value for determining whether there is a risk of onset of dementia, but not higher than a cutoff value for determining whether there is incidence of dementia. Alternatively, for example, the degree of the risk of onset may be classified into a plurality of levels, and a cutoff value or a control value may be set for each level.

The method of the present aspect encompasses a method not only when the method uses one of the cutoff value or the control value, but also when the method uses both in combination.

The type of the determination result in the present step is not particularly limited. For example, as described above, the result may be used for determining whether there is incidence of dementia or whether there is a risk of onset of dementia, as well as for determining the severity of dementia in a subject. Further, the type of dementia to be subjected to determination is also not particularly limited. For example, the type of the dementia includes Alzheimer's-type dementia, dementia with Lewy bodies, and vascular dementia.

The method of one or more embodiments of the present invention may also be used in combination with known biomarkers for testing dementia, such as amyloid-β protein and tau protein, and imaging tests such as PET that enable visualization of such biomarkers. When the present step is included, the method of the present aspect may be used as a determination method, or as a method that assists a physician in making the determination.

### 2-3. Effect

The method of the present aspect employs a less-invasive, simple operation to allow determination of whether there is incidence of dementia and whether there is a risk of onset of dementia with high sensitivity and specificity.

Further, the method of the present aspect enables judgment of whether intervention is necessary to prevent, improve, or treat dementia. When such a judgment is made, the method of the present aspect may be used as a method for selecting a target animal individual to be treated with an active substance for the improvement, treatment, and/or prevention of dementia.

### 3. Kit

### 3-1. Overview

A third aspect of one or more embodiments of the present invention is a kit for determining whether there is incidence of dementia or whether there is a risk of onset of dementia. By the kit of the present aspect, the biomarker according to the first aspect can be detected, and/or the amount of the biomarker can be measured.

### 3-2. Constitution

The kit of one or more embodiments of the present invention comprises, as an essential component, a reagent or a peptide-binding molecule for measuring the gene expression level.

When the kit of one or more embodiments of the present invention comprises a reagent for measuring the gene expression level, the kit comprises a reagent for measuring the expression level of the Cx37 gene in a sample derived from a subject, and a reagent(s) for measuring the expression level(s) of the Cx43 gene and/or the PHD3 gene in the sample.

On the other hand, when the kit of one or more embodiments of the present invention comprises a peptide-binding molecule, the kit comprises a binding molecule for Cx37 in a sample derived from a subject, and a binding molecule for Cx43 and/or a binding molecule for PHD3 in the sample.

The reagents and the binding molecules are specifically described below.

### 3-2-1. Reagent for Measuring Gene Expression Level

The reagent for measuring the gene expression level, used in the kit of one or more embodiments of the invention, refers to a molecule that specifically binds to a polynucleotide or a fragment thereof in the biomarker according to the first aspect. Specifically, the reagent refers to a primer capable of amplifying all or part of the base sequence of the target gene or the complementary base sequence thereto; and/or a probe capable of hybridizing with all or part of the base sequence of the target gene or the complementary base sequence thereto.

The polynucleotide in the biomarker, that is, the target nucleotide, herein refers to the Cx37 gene or the like/Cx43 gene or the like, and the Cx37 gene or the like/PHD3 gene or the like.

The base sequences of the primer and the probe in the kit of one or more embodiments of the present invention are not particularly limited as long as they have the functions described above. For example, the primer and the probe include the following polynucleotides:
(1) a fragment consisting of 15 or more consecutive nucleotides selected from a polynucleotide consisting of the base sequence according to SEQ ID NO:1, 3 or 5;
(2) a polynucleotide fragment having deletion, substitution, addition, or insertion of 1 or 2 bases in the base sequence of the polynucleotide fragment of (1);
(3) a fragment comprising 15 or more consecutive nucleotides selected from a polynucleotide consisting of the base sequence according to SEQ ID NO:1, 3 or 5;
(4) a polynucleotide fragment having deletion, substitution, addition, or insertion of 1 or 2 bases in the base sequence of the polynucleotide fragment of (3);
(5) a polynucleotide that hybridizes under highly stringent conditions with a fragment sequence comprising 15 or more consecutive nucleotides selected from the base sequence according to SEQ ID NO:1, 3, or 5; and
(6) a polynucleotide consisting of the base sequence complementary to the base sequence of the polynucleotide or a fragment thereof of (1) to (5).

The term “probe” herein encompasses a polynucleotide capable of specifically recognizing and detecting RNA generated by gene expression or a polynucleotide derived therefrom, a polynucleotide complementary thereto, and an aptamer.

The term “primer” herein encompasses a polynucleotide capable of specifically recognizing and amplifying RNA generated by gene expression or a polynucleotide derived therefrom, and a polynucleotide complementary thereto.

“Complementary” refers to a relationship in which nucleic acid bases can form base pairs with each other through hydrogen bonds. The so-called Watson-Crick base pairs (natural base pairs) or Hoogsteen base pairs correspond to such a relationship.

The term “hybridize” or “capable of hybridizing” herein means that base pairing occurs between polynucleotides having base sequences complementary to each other, to form a fully or partially complementary double strand.

The term “stringent conditions” herein refers to conditions under which a nucleic acid probe hybridizes with its target sequence to a greater extent than with other sequences (for example, the measured value is more than “the average of measured background values+the standard error of the measured background values×2”). The stringent conditions are sequence-dependent and vary depending on the environment for the hybridization.

The term “highly stringent condition” herein refers to environmental conditions under which non-specific hybridization hardly occurs. Under highly stringent conditions, a hybrid can be formed with a nucleic acid having a target base sequence, but a hybrid cannot be substantially formed with a nucleic acid having a non-specific base sequence. In general, “highly stringent conditions” refers to conditions with low salt concentration and high temperature. “Low salt concentration” refers to, for example, 15 to 750 mM, particularly 15 to 500 mM, 15 to 300 mM, or 15 to 200 mM. For example, 1.5 to 3.5×SSC, 2 to 3×SSC, or 2 to 2.5×SSC is comprised. Further, “high temperature” refers to, for example, 50 to 68° C., or 55 to 70° C. Another condition that defines the stringent conditions is the washing conditions after the hybridization. For example, the washing conditions include washing at 65° C. in 0.1×SSC and 0.1% SDS after the hybridization. The polynucleotide contained in the kit of one or more embodiments of the present invention particularly maintains the hybridized state with the target plus strand even after washing under such conditions. Specific examples of the polynucleotide include a polynucleotide consisting of the base sequence fully complementary to the base sequence of the target plus strand, and a polynucleotide consisting of a base sequence having an identity of 80% or more, 85% or more, 90% or more, 95% or more, 98% or more, or 99% or more to the above polynucleotide.

Other examples of “stringent conditions” in these hybridizations are described in, for example, Sambrook, J. & Russel, D. (authors), Molecular Cloning, A LABORATORY MANUAL, Cold Spring Harbor Laboratory Press, published on Jan. 15, 2001, vol. 1, 7.42 to 7.45, vol. 2, 8.9 to 8.17. These conditions may be used in one or more embodiments of the present invention.

The value of the identity represents a value calculated using software for the calculation of the identity between a plurality of base sequences (for example, FASTA, DNASIS, and BLAST) with default settings. The value of the base sequence identity is calculated by aligning a pair of base sequences to achieve the maximum degree of matching, calculating the number of the resulting matching bases, and then determining the proportion of the number of the matching bases to the total number of bases in the base sequence compared. Here, when a gap(s) is/are present, each gap is counted as 1 base to determine the total number of bases described above. For details of the method for determining the identity, see, for example, Altschul et al, Nuc. Acids. Res. 25, 3389-3402, 1977; and Altschul et al, J. Mol. Biol. 215, 403-410, 1990.

The length of the consecutive nucleotides in each of these polynucleotides is not particularly limited as long as it is 15 bases or more. For example, the length may be 17 bases or more, 18 bases or more, 19 bases or more, 20 bases or more, or 21 bases or more. The level of identity is particularly 80% or more, 90% or more, 95% or more, 96% or more, 97% or more, 98% or more, or 99% or more.

Further, each of these polynucleotides may also be a polynucleotide comprising a polynucleotide that comprises a base sequence having not less than a certain degree of identity with a continuous partial base sequence contained in the base sequence of the target gene, or with the complementary base sequence thereto.

When a primer is used, the primer particularly comprises the above-described consecutive nucleotides in its 3′-end side, which is the direction of extension. Further, the primer particularly comprises, in the order from the 3′-end, a base sequence region consisting of a base sequence with an identity of 100% to the target base sequence, and a base sequence region consisting of a base sequence having not less than a certain level of identity to the target base sequence. The length of the base sequence region consisting of the identical base sequence as the target base sequence is not particularly limited, and is, for example, 2 bases or more, 3 bases or more, 5 bases or more, 10 bases or more, 15 bases or more, 17 bases or more, or 19 bases or more.

When such a primer is used, it may comprise an optional additional base sequence in its 5′-end side. The number of bases of the entire primer is not particularly limited, and may be, for example, 50 bases or less, 40 bases or less, or 30 bases or less.

For example, the specific primer pairs comprised in the kit of one or more embodiments of the present invention include, for the Cx37 gene, a pair of polynucleotides comprising, or consisting of, the base sequences of SEQ ID NOs: 9 and 10; for the Cx43 gene, a pair of polynucleotides comprising, or consisting of, the base sequences of SEQ ID NOs: 11 and 12; and, for the PHD3 gene, a pair of polynucleotides comprising, or consisting of, the base sequences of SEQ ID NOs: 17 and 18.

For example, conditions for carrying out the PCR using the primers in the kit of one or more embodiments of the present invention include treatment using a PCR buffer having a composition such as 10 mM Tris-HCl (pH 8.3), 50 mM KCl, 1 to 2 mM MgCl₂, at a Tm value calculated from the primer sequence+5 to 10° C. for about 15 seconds to 1 minute. For example, the calculation method for the Tm value includes the following: Tm value=2×(number of adenine residues+number of thymine residues)+4×(number of guanine residues+number of cytosine residues).

The nucleic acid constituting the probe contained in the kit of one or more embodiments of the present invention for use in the hybridization method may be DNA since it can usually be synthesized at low cost and is stable. However, when necessary, all or part of the nucleic acid may comprise a chemically modified nucleic acid such as PNA (Peptide Nucleic Acid), BNA (Bridged Nucleic Acid)/LNA (Locked Nucleic Acid), methylphosphonate-type DNA, phosphorothioate-type DNA, or 2′-O-methyl-type RNA; pseudo nucleic acid; or a combination thereof. Further, the probe contained in the kit of one or more embodiments of the present invention may be modified or labeled using a fluorescent dye (for example, fluorescamine or a derivative thereof, rhodamine or a derivative thereof, DIG, FITC, Cy3, Cy5, FAM, HEX, or VIC); a quencher substance (TAMRA, DABCYL, BHQ-1, BHQ-2, or BHQ-3); a modifying substance such as biotin or (strept) avidin, or magnetic beads; a radioisotope (for example, ³²P, ³³P, ³⁵S); or the like. The hybridization is particularly carried out under stringent conditions at low salt concentration and high temperature in order to eliminate non-target nucleic acid that non-specifically hybridizes.

Each polynucleotide or fragment thereof used in the kit of one or more embodiments of the present invention may be either DNA or RNA.

The polynucleotide used in the kit of one or more embodiments of the present invention may be prepared using a common technique, such as the DNA recombination technique, the PCR method, or a method by an automated DNA/RNA synthesizer.

The DNA recombination technique and the PCR method may be carried out using a technique described in, for example, Ausubel et al., Current Protocols in Molecular Biology, John Willey & Sons, US (1993); or Sambrook et al., Molecular Cloning A Laboratory Manual, Cold Spring Harbor Laboratory Press, US (1989).

The target base sequence is a known base sequence, and a method for obtaining it is also known as described above. Therefore, a polynucleotide as a probe or a primer that can be used for the kit of one or more embodiments of the present invention may be prepared by cloning this gene.

Such a probe or primer may be chemically synthesized using an automated DNA synthesizer. In general, this synthesis employs the phosphoramidite method, and this method enables automated synthesis of a single-stranded DNA of up to about 100 bases. Automated DNA synthesizers are commercially available from, for example, Polygen, ABI, or Thermo Fishers. Alternatively, the probe or primer may be prepared by the cDNA cloning method.

The kit of one or more embodiments of the present invention may comprise a probe and/or a primer in any combination. The kit usually comprises the primer as the primer pair of a forward primer and a reverse primer. Further, the kit particularly comprises at least one of a primer set for the detection of the biomarker combination according to the first aspect, or a probe set for the detection of the biomarker combination according to the first aspect.

The kit of one or more embodiments of the present invention may additionally comprise a probe and/or a primer for any known gene that may be used as a biomarker capable of determining whether there is incidence of dementia or whether there is a risk of onset of dementia.

### 3-2-2. Peptide-Binding Molecule

The term “peptide-binding molecule” herein refers to a molecule that specifically binds to a polypeptide or a peptide fragment thereof in the biomarker according to the first aspect and specifically refers to an antibody or an active fragment thereof, or aptamer, capable of binding to the target protein.

The polypeptide in the biomarker, that is, the target nucleotide, herein refers to the Cx37 or the like/Cx43 or the like, and the Cx37 or the like/PHD3 or the like.

The kit of one or more embodiments of the present invention comprises one or more peptide-binding molecules that bind to these polypeptides. The peptide-binding molecule may be any of a peptide, a nucleic acid, a low molecular weight compound, or a combination thereof.

### 3-2-3. Other Constitutions

The kit of one or more embodiments of the present invention may also comprise a reagent for extracting a nucleic acid (such as total RNA) or a polypeptide from a body fluid, a cell or a tissue; a fluorescent substance for labeling; an enzyme for nucleic acid amplification, and a culture medium; instructions for use; a dilution or reaction buffer comprising a component required for the measurement; a washing solution; a coloring reagent; a reaction container; or the like.

The kit of one or more embodiments of the present invention may further comprise a reagent and/or a column for separating mononuclear cells from the sample. For example, the reagent and/or the column include Ficoll-Paque PLUS (manufactured by GE Healthcare), Lymphoprep (manufactured by Abbott Diagnostics Technologies), the Human Peripheral Blood Mononuclear Cell Isolation and Viability Kit (manufactured by BioVision), the BD Vacutainer (registered trademark) CPT blood collection tube for separation of mononuclear cells (manufactured by Becton Dickinson), pluriMate (manufactured by pluriSelect), and SepMate (manufactured by STEMCELL Technologies). These enable separation of mononuclear cells and granulocytes in the sample from each other, to allow efficient acquisition of the mononuclear cell-comprising sample to be subjected to the measurement.

### 4. Device

### 4-1. Overview

A fourth aspect of one or more embodiments of the present invention is a device for determining whether there is incidence of dementia or whether there is a risk of onset of dementia. By the device of the present aspect, the biomarker according to the first aspect can be detected, and/or the amount of the biomarker can be measured.

### 4-2. Constitution

The device of one or more embodiments of the present invention comprises, as an essential component, a reagent or a peptide-binding molecule for measuring the gene expression level.

When the device of one or more embodiments of the present invention comprises a reagent for measuring the gene expression level, the device of comprises a reagent for measuring the expression level of the Cx37 gene in a sample derived from a subject, and a reagent(s) for measuring the expression level(s) of the Cx43 gene and/or the PHD3 gene in the sample.

On the other hand, when the device of one or more embodiments of the present invention comprises a peptide-binding molecule, the kit of one or more embodiments of the present invention comprises a binding molecule for Cx37 in a sample derived from a subject, and a binding molecule for Cx43 and/or a binding molecule for PHD3 in the sample.

Regarding the reagent and the peptide-binding molecule used to measure the expression level, a detailed description was given for the third aspect. Therefore, their description is omitted here.

The other constitutions are basically in accordance with the description of the third aspect.

When necessary, the device of one or more embodiments of the present invention may additionally comprise means for collecting a sample from the subject, means for measuring the amount of the biomarker, means for calculating the ratio, and the like.

The device of one or more embodiments of the present invention is a device for measuring a biomarker, wherein the above-described nucleic acid in one or more embodiments of the present invention such as a polynucleotide, a mutant thereof, a derivative thereof, or a fragment thereof is bound or attached to a solid phase. The material of the solid phase is, for example, plastic, paper, glass, or silicone. From the perspective of ease of processing, plastic is particularly suitable as the material of the solid phase. The solid phase may have any shape such as a square shape, a round shape, a strip-like shape, or a film-like shape. For example, the device of one or more embodiments of the present invention includes devices for measurement by the hybridization technique, specifically, blotting devices and nucleic acid arrays (such as microarrays, DNA chips, and RNA chips).

In the nucleic acid array technique, the above-described nucleic acid is bound or attached, one by one, to a surface of a solid phase that has been subjected, when necessary, to surface treatment such as L-lysine coating or introduction of functional groups, for example, amino groups, carboxyl groups, or the like, to prepare an array such as a chip, and the array is then used for measurement of the target nucleic acid utilizing hybridization. For example, the method for binding or attaching the nucleic acid includes a method in which the nucleic acid is spotted using a high-density dispenser called a spotter or an arrayer, a method in which the nucleic acid is sprayed to the solid phase using an inkjet that ejects very small droplets from a nozzle using a piezoelectric element or the like, or a method in which nucleotide synthesis is allowed to proceed sequentially on a solid phase.

### 5. Method for Screening Candidate Substance or Therapeutic Conditions that May be Effective in Improvement, Treatment, and/or Prevention of Dementia

### 5-1. Overview

A fifth aspect of one or more embodiments of the present invention is a method for screening a candidate substance or therapeutic conditions that may be effective in improvement, treatment, and/or prevention of dementia. By the method of the present aspect, a candidate substance for an agent or therapeutic conditions useful for the treatment and the like of dementia can be screened based on the biomarker according to the first aspect.

### 5-2. Method

The method of one or more embodiments of the present invention comprises, as essential steps, a step of exposure to a test substance or test conditions, a step of measuring the amount of a biomarker, a step of calculating the ratio, and a step of determination.

### 5-2-1. Step of Exposure to Test Substance or Test Conditions

The present step is a step of exposing a cell to a test substance or test conditions.

The term “therapeutic conditions” herein refers to treatment conditions used to improve, treat, and/or prevent dementia. The term “test conditions” herein refers to treatment conditions that may be used to improve, treat, and/or prevent dementia. For example, when an apparatus such as a medical device is applied, or when exercise therapy or the like is carried out as a treatment, “treatment conditions” refers to the conditions under which the apparatus is applied, or under which the therapy or the like is carried out. “Exposure to test conditions” refers to the application of test conditions to the target of exposure.

The type of the cell is not particularly limited, and the cell is particularly a cell contained in the sample in the present specification. Specifically, for example, the cell includes cells contained in body fluids, cells contained in bone marrow, and mononuclear cells. The cell may be a cell in the body or may be an isolated cell. When a cell in the body is used as the above-described cell, the cell is particularly a cell in the body of a non-human animal. Further, when an isolated cell is used, the method of the present aspect is an in vitro method. The cell used in the method of the present aspect is a cell whose change due to the exposure can be an index for determining whether the test substance or the test conditions is/are effective for the treatment and/or prevention of dementia. Specifically, for example, a cell derived from an individual suffering from incidence of dementia may be used in the method of the present aspect.

The method of exposure to the test substance in the present step is not particularly limited. The method may be appropriately determined according to the properties of the test substance, the cell type, and the like. The method of exposure to the test conditions in the present step is not particularly limited. The method is appropriately determined according to the type and the properties of the phenomenon utilized, the type of apparatus or equipment used, the type of treatment means (for example, increased amount of exercise), and the like.

For example, when the target of exposure is an isolated cell, the cell may be exposed by incubating it in the presence of the test substance or test conditions. Further, when the target of exposure is a cell in the body of a non-human animal, the test substance or the test conditions is/are administered or applied to the non-human animal.

The method of administration of the test substance is not particularly limited and may be either oral administration or parenteral administration.

The specific method for applying or carrying out the test conditions is not particularly limited. For example, when an apparatus such as a medical device is used, various parameters such as the application target area, the amount of energy (output) to be applied, the length of time, the number of times, and the duration are appropriately determined according to the type and the like of the target individual or cell. Further, the application method is not particularly limited also when the method comprises a treatment without the use of any apparatus, such as exercise therapy. For example, when exercise therapy is carried out, various parameters such as the part to be moved, the speed, the range, the amount of load, the degree of difficulty, the length of time, the number of times, and the duration are appropriately determined according to the specific means such as the type of the exercise, the health condition of the subject individual, and the like.

### 5-2-2. Step of Measuring Amount of Biomarker

The present step is a step of measuring the amount of the biomarker according to the first aspect in a cell. The present step may be carried out at the same time as or after the step of exposure to the test substance or test conditions.

Details of the present step are in accordance with the “Step of Measuring Amount of Biomarker” in the second aspect. Therefore, only the differences are described here.

When the cell as the target of exposure in the step of exposure to the test substance or test conditions is a cell in the body of a non-human animal, a sample may be isolated from the non-human animal, and the amount of the biomarker may be measured in the isolated sample in the present step. The method for isolating the sample is not particularly limited.

### 5-2-3. Step of Calculating Ratio

The present step is a step of calculating the ratio between the expression level of the Cx37 gene or the amount of protein of Cx37, and the expression level of another gene or the amount of another protein. The present step may be carried out at the same time as or after the step of measuring the amount of the biomarker.

Details of the present step are in accordance with the “Step of Calculating Ratio” in the second aspect. Therefore, a detailed description is omitted here.

### 5-2-4 Step of Determination

The present step is a step of determining, based on a relative value, whether the test substance or the test conditions is/are effective for the improvement, treatment, and/or prevention of dementia. The present step may be carried out at the same time as or after the step of calculating the relative value of the expression level.

Details of the present step are basically in accordance with the “Step of Determination” in the second aspect. Therefore, only the differences are described here.

In the present step, the test substance or test conditions is/are determined to be a candidate substance or therapeutic conditions effective for the improvement, treatment, and/or prevention of dementia when the relative value of the expression level of the Cx37 gene or of the amount of protein of Cx37 is lower than a cutoff value, or when the relative value of the expression level of the Cx37 gene or of the amount of protein of Cx37 is significantly lower than a control value.

Alternatively, the test substance or test conditions is/are determined to be a candidate substance or therapeutic conditions effective for the improvement, treatment, and/or prevention of dementia when the relative value to the expression level of the Cx37 gene or to the amount of protein of Cx37 is higher than a cutoff value, or when the relative value to the expression level of the Cx37 gene or to the amount of protein of Cx37 is significantly higher than a control value.

### EXAMPLES

### Example 1. Measurement of Expression Level of Connexin Gene in Dementia Patient

### (Object)

The difference in the expression level of the connexin gene between dementia patients and healthy individuals is investigated.

### (Methods)

### 1. Selection of Individuals

From males with an age of 65 or older, which is the defining age for young-onset dementia, 10 volunteers for each were selected as healthy persons and dementia patients.
Preexisting diseases, complications, and the like to be excluded: diabetes, active cancer diseases, receiving treatment for a cancer disease, less than 5 years after the completion of cancer treatment, thyroid disease, infectious disease (AIDS, hepatitis B or C), fever at the time of blood collection, chronic subdural hematoma, normal pressure hydrocephalus, receiving hemodialysis, scheduled for hemodialysis (being informed by a physician that hemodialysis will be required in the near future), COPD, receiving home oxygen therapy, smoking within the past 12 months
Medication status to be excluded: agents that affect the immune system, such as anticancer drugs, steroids, antirheumatic drugs
Incidence of dementia: regarding dementia patients, individuals whose diagnosis has been confirmed by radiologist's observation and the like based on MRI, SPECT examination, or the like at a medical facility.
MMSE test: 28 points or more regarding healthy persons; 23 points or less regarding dementia patients

### 2. Examination and Blood Collection

In accordance with informed consent, the purpose of use and the like of the obtained specimen was explained to each individual, and consent was obtained prior to the examination and blood collection. For healthy persons, consent was obtained from themselves. For dementia patients, consent was obtained from themselves and/or their guardians.

When no MMSE test has been carried out within 3 months, an MMSE test was performed at the time when the consent was obtained.

The patient was fasted from the previous night, and blood was collected in the morning.

The blood was collected from the forearm cutaneous vein using a 10-mL syringe with a 21G needle.

A total of 8 mL to 8.5 mL of blood was collected, and 2.5 mL of the collected blood was dispensed into a PAXgene® RNA blood collection tube (manufactured by Becton-Dickinson) as a sample for gene analysis of whole blood. As a sample for the peripheral blood picture, 0.5 mL to 1 mL of the blood was dispensed into a blood collection tube containing EDTA. In addition, 4 mL of the blood was dispensed into a CPT™ blood collection tube for mononuclear cell separation (manufactured by Becton-Dickinson) as a sample for the preparation of a mononuclear cell suspension. The sample for gene analysis was stored at room temperature for 2 hours or more, and then cryopreserved until the sample was used for gene expression analysis.

The proportion of mononuclear cells was calculated as follows. The sample for the peripheral blood picture was visually inspected under the microscope (microscopy), to count the numbers of band neutrophils, segmented neutrophils, lymphocytes, monocytes, eosinophils, and basophils in the whole-blood sample. The proportion of mononuclear cells (lymphocytes and monocytes) in the total blood cells counted as above was calculated as the proportion of mononuclear cells.

### 3. Preparation of Mononuclear Cell Suspension

The sample for the preparation of a mononuclear cell suspension was centrifuged at 1800×g at room temperature for 20 minutes. After gentle mixing by inversion, 2.5 mL of the supernatant was collected using a 2.5-mL syringe with a 21G needle as a mononuclear cell suspension.

The mononuclear cell suspension and the remaining liquid were placed in separate PAXgene® RNA blood collection tubes (manufactured by Becton-Dickinson) and used as samples for gene analysis. Each sample for gene analysis was stored at room temperature for 2 hours or more and then cryopreserved until the sample was used for gene expression analysis.

### 4. Gene Expression Analysis

First, total RNA was extracted from the sample for gene expression analysis. For the extraction, an RNA extraction kit (RiboPure Blood RNA Isolation Kit (Thermo Fisher Scientific, catalog number: AM1928)) was used according to the manufacturer's protocol.

Subsequently, cDNA was synthesized (reverse-transcribed) from 1 μg of the total RNA. For the cDNA synthesis, a cDNA synthesis kit (PrimeScript® II 1 st strand cDNA Synthesis Kit (Takara Bio Inc., catalog number: 6210B (A×4))) was used according to the manufacturer's protocol.

Real-time PCR reaction was carried out using the obtained cDNA as a template. As a nucleic acid amplification reagent, PowerUp SYBR® Green Master Mix (Thermo Fisher Scientific, catalog number: A25777) was used. Further, as a real-time PCR system, Agilent AriaMx Real-Time PCR System (Agilent) was used. The real-time PCR reaction conditions were as follows: 1 cycle at 50° C. for 3 minutes; 1 cycle at 95° C. for 3 minutes; 40 cycles at 95° C. for 5 seconds and at 60° C. for 30 seconds; 1 cycle at 95° C. for 30 seconds; 1 cycle at 65° C. for 30 seconds; and 1 cycle at 95° C. for 30 seconds.

The 18S ribosomal RNA gene, which is a housekeeping gene, was used as an endogenous control, and the relative expression level of each gene to the endogenous control was analyzed by the Pfaffl method. Specifically, the Pfaffl method is a relative quantification method that is a type of the comparative Ct method, in which the PCR amplification efficiencies of a housekeeping gene and a gene are taken into account (Pfaffl M W. Nucleic Acids Res. 2001; 29(9): e45).

Table 1 shows the base sequences of the primers for each gene. 18S represents 18S ribosomal RNA; Glut1 and Glut3 represent glucose transporters 1 and 3, respectively; MCT4 represents monocarboxylate transport protein 4; and PDK1 represents pyruvate dehydrogenase kinase 1. Further, Fw represents a forward primer, and Rv represents a reverse primer.

TABLE 1
Primers used in Examples

| gene | base sequence (5′→3′) | |
|---|---|---|
| 18S | Fw | ACTCAACACGGGAAACCTCACC |
| | | (SEQ ID NO: 7) |
| | Rv | CCAGACAAATCGCTCCACCA |
| | | (SEQ ID NO: 8) |
| Cx37 | Fw | CACCATGCCCCACCTACAAT |
| | | (SEQ ID NO: 9) |
| | Rv | TGGGGGTTTTTGGCCATTCT |
| | | (SEQ ID NO: 10) |
| Cx43 | Fw | AGGAGTTCAATCACTTGGCGT |
| | | (SEQ ID NO: 11) |
| | Rv | CCCTCCAGCAGTTGAGTAGG |
| | | (SEQ ID NO: 12) |
| Glut1 | Fw | CCTGCAGTTTGGCTACAACAC |
| | | (SEQ ID NO: 13) |
| | Rv | CAGGATGCTCTCCCCATAGC |
| | | (SEQ ID NO: 14) |
| Glut3 | Fw | ATTACAGCGATGGGGACACA |
| | | (SEQ ID NO: 15) |
| | Rv | GCCAAATTGGAAAGAGCCGA |
| | | (SEQ ID NO: 16) |
| MCT4 | Fw | CGGAGCATCATCCAGGTCTAC |
| | | (SEQ ID NO: 21) |
| | Rv | GGCTGGAAGTTGAGTGCCAA |
| | | (SEQ ID NO: 22) |
| PHD3 | Fw | GATCGTAGGAACCCACACGA |
| | | (SEQ ID NO: 17) |
| | Rv | TCAGAGCACGGTCAGTCTTC |
| | | (SEQ ID NO: 18) |
| PDK1 | Fw | GCAAAATCACCAGGACAGCC |
| | | (SEQ ID NO: 19) |
| | Rv | TCTGTTGGCATGGTGTTCCA |
| | | (SEQ ID NO: 20) |

The free statistical software EZR (Easy R version 1.54) was used to determine the cutoff value, and to calculate determination accuracies such as the sensitivity, specificity, and AUC. The Youden index was used to determine the cutoff value.

The Welch's t-test was used to calculate the significant difference.

### (Results)

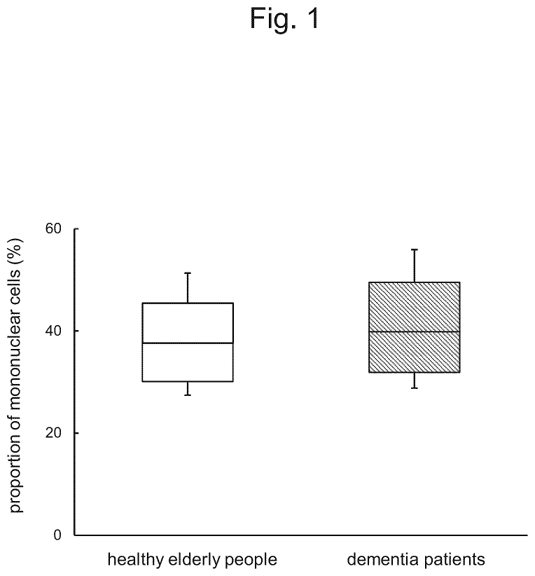

The results are shown in FIGS. 1 to 3.

First, the proportion of mononuclear cells was not significantly different between the healthy subjects and the dementia patients (FIG. 1).

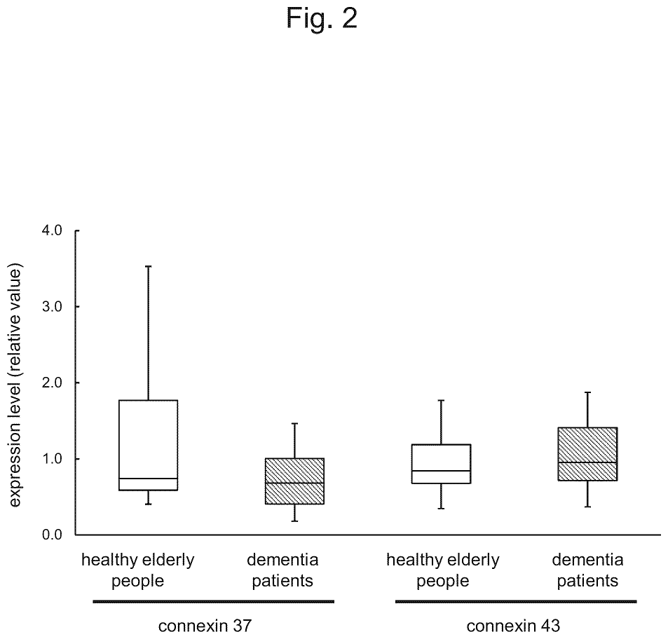

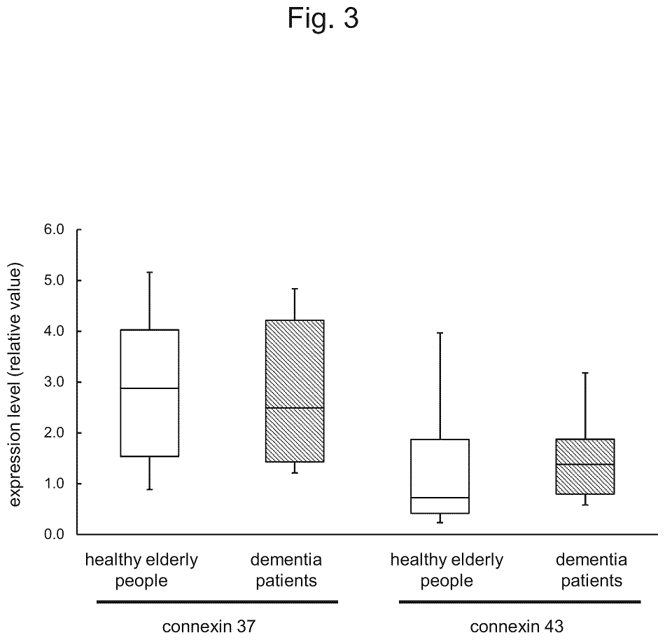

The expression levels of the connexin 37 gene and the connexin 43 gene were not significantly different between the healthy persons and the dementia patients in either the mononuclear cell-comprising sample (FIG. 2) or the whole-blood sample (FIG. 3). However, regarding the connexin 37 gene in both samples, the expression level tended to be slightly lower in dementia patients than in healthy subjects. Additionally, in contrast, regarding the connexin 43 gene, the expression level tended to be slightly higher in dementia patients. Further, when the connexin genes were used, the determination accuracies for dementia patients were not high.

Thus, the expression level of each connexin gene in peripheral blood was found not to be sufficiently applicable as a biomarker for the incidence of dementia when it is used alone.

### Example 2. Test of Accuracy of the Determination of Dementia Using Each of Various Biomarkers Alone

### (Object)

The accuracy of determination of dementia using each of the biomarkers found in WO 2021/177447 alone is investigated.

### (Methods)

Using the analysis results on the gene expression levels in Example 1, the determination accuracies were calculated by the same method as in Example 1.

### (Results)

The results are shown in Tables 2 and 3.

TABLE 2
Dementia determination accuracy achieved using each biomarker
alone in mononuclear cell-comprising samples

| | PHD3 | PDK1 | GLUT1 | GLUT3 | MCT4 |
|---|---|---|---|---|---|
| specificity (%) | 80 | 90 | 50 | 80 | 70 |
| sensitivity (%) | 50 | 30 | 80 | 50 | 50 |
| AUC | 0.70 | 0.55 | 0.66 | 0.57 | 0.49 |

TABLE 3
Dementia determination accuracy achieved using
each biomarker alone in whole-blood samples

| | PHD3 | PDK1 | GLUT1 | GLUT3 | MCT4 |
|---|---|---|---|---|---|
| specificity (%) | 50 | 60 | 80 | 50 | 80 |
| sensitivity (%) | 90 | 70 | 60 | 80 | 60 |
| AUC | 0.72 | 0.61 | 0.68 | 0.61 | 0.66 |

Table 2 shows the accuracy of the determination of dementia patients as observed using each biomarker alone in the mononuclear cell-comprising samples. Table 3 shows the accuracy of the determination of dementia patients as observed using each biomarker alone in the whole-blood samples.

In none of the markers, the sensitivity and the specificity did not exceed 70% at the same time. Further, AUC, which is a value indicating the judgment ability of an index, was highest for the prolyl hydroxylase domain-containing protein 3 (PHD3) gene, but the value was still only around 0.7.

Thus, the expression levels of the known biomarker genes, when each of these was used alone, were found not to be capable of achieving a determination accuracy sufficient for the definitive diagnosis in dementia patients.

### Example 3. Test of Accuracies of the Determination of Dementia Using Relative Values of Expression Level of Connexin 37 Gene to Expression Levels of Various Biomarker Genes

### (Object)

The accuracies of determination of dementia achieved when relative values of the expression level of the connexin 37 gene to the expression levels of connexin 43 and the biomarker genes investigated in Example 2 are used are investigated.

### (Methods)

Using the analysis results on the gene expression levels in Example 1, determination accuracies were calculated by the same method as in Example 1.

The relative value was calculated by dividing the expression level of the connexin 37 gene by the expression level of each of the other genes.

### (Results)

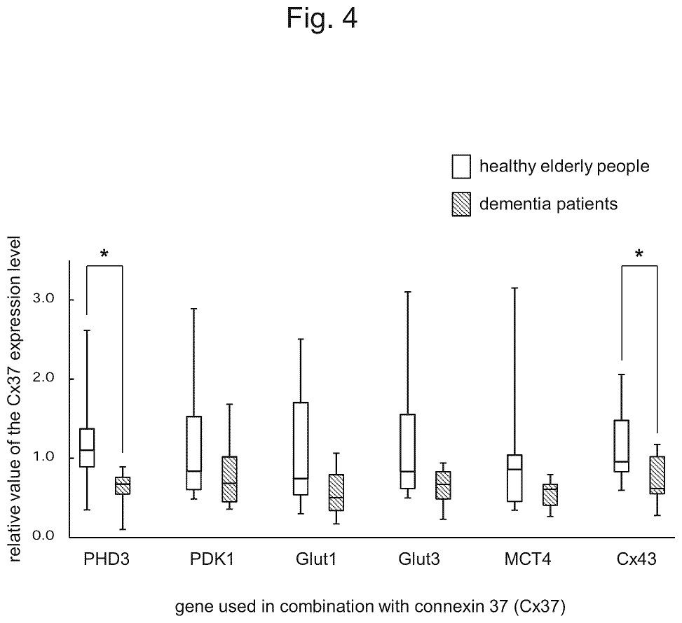

The results are shown in FIG. 4 and Table 4.

TABLE 4
Dementia determination accuracy of the relative value
of the expression level of the connexin 37 gene

| | gene used in combination with connexin 37 (Cx37) | | | | | |
|---|---|---|---|---|---|---|
| | PHD3 | PDK1 | GLUT1 | GLUT3 | MCT4 | Cx43 |
| specificity (%) | 80 | 50 | 40 | 40 | 60 | 90 |
| sensitivity (%) | 100 | 70 | 100 | 90 | 90 | 70 |
| AUC | 0.88 | 0.67 | 0.73 | 0.71 | 0.69 | 0.79 |

Table 4 shows the accuracies of the determination of dementia patients as observed using each relative value in the mononuclear cell-comprising samples.

When the connexin 37 gene was used in combination with each gene, all combinations generally showed improvement in the determination accuracy compared to those observed when each gene expression level was used alone. In particular, when the PHD3 gene was used in combination (cutoff value: 0.893) and when the connexin 43 gene was used in combination (cutoff value: 0.651), both the sensitivity and the specificity were higher than 70%, and the AUC was well above 0.75.

The same results were also obtained when the values were compared between healthy subjects and dementia patients (FIG. 4). As shown in FIG. 4, among the combinations investigated, the relative value was significantly lower in dementia patients than in healthy subjects only when the expression level of the connexin 37 gene was used in combination with the expression level of the PHD3 gene or the connexin 43 gene.

Thus, the relative value of the expression level of the connexin 37 gene to the expression level of the PHD3 gene or the connexin 43 gene was found to be an excellent marker whose determination accuracy for dementia patients is sufficiently applicable to definitive diagnosis.

### Example 4. Test of Accuracy of the Determination of Dementia Using Relative Value of Expression Level of Connexin 37 Gene to Expression Level of PHD3 Gene

### (Object)

The accuracy of determination of dementia achieved when a relative value of the expression level of the connexin 37 gene to the expression level of the PHD3 gene is used is investigated.

### (Methods)

Using the analysis results on the gene expression levels in Example 1, determination accuracies were calculated by the same method as in Example 1.

The relative value was calculated by dividing the expression level of the connexin 37 gene by the expression level of the PHD3 gene.

### (Results)

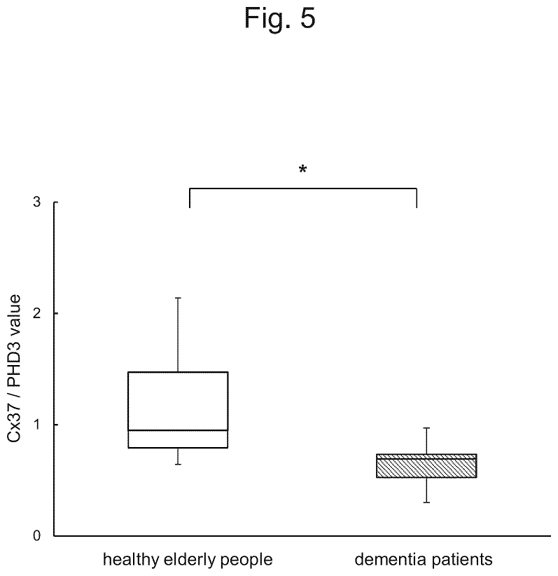

The results are shown in FIG. 5.

Regarding the accuracy of the determination of dementia patients by the relative value to the expression level of the PHD3 gene in the whole-blood samples, both the sensitivity and the specificity were 90%, and the AUC was 0.9 (cutoff value: 0.744), indicating that this is an extremely excellent biomarker.

The same result was obtained also when the value was compared between healthy subjects and dementia patients (FIG. 5). As shown in FIG. 5, in accordance with the results obtained when the mononuclear cell-comprising samples were used, the whole-blood samples also showed significantly lower relative values of the expression level of the connexin 37 gene to the expression level of the PHD3 gene in dementia patients compared to healthy subjects.

Thus, the relative value of the expression level of the connexin 37 gene which was useful in the mononuclear cell-comprising samples, was also found to be useful in the whole-blood samples.

### Example 5. Test of Accuracy of the Determination of Dementia Using Relative Value to Expression Level of Connexin 37 Gene

### (Object)

The accuracy of the determination of dementia achieved when the numerator and the denominator are inverted, specifically, when a relative value of the expression level of the PHD3 gene to the expression level of the connexin 37 gene is used is investigated.

### (Methods)

Using the analysis results on the gene expression levels in Example 1, determination accuracies were calculated by the same method as in Example 1.

The relative value was calculated by dividing the expression level of the PHD3 gene by the expression level of the connexin 37 gene.

### (Results)

When the relative value to the connexin 37 gene was used, both in the mononuclear cell-comprising samples and in the whole-blood samples, the accuracy of the determination of dementia patients was completely the same as those found when the relative value of the expression level of the connexin 37 gene was used, regarding all of the sensitivity, specificity, and AUC (cutoff value: mononuclear cell-comprising samples, 1.12; whole-blood samples, 1.344).

Thus, the ratio between the expression level of the connexin 37 gene and the expression level of another gene such as the PHD3 gene could be confirmed to be useful in the determination of dementia irrespective of which expression level is used as the denominator.

All publications, patents, and patent applications cited herein are hereby incorporated by reference in their entirety.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

1. A method for determining whether there is incidence of dementia or whether there is a risk of onset of dementia in a subject,
wherein the method comprises:
measuring gene expression levels of connexin 37 and connexin 43 and/or prolyl hydroxylase domain-containing protein 3 in a sample derived from the subject; and
calculating a ratio(s) between gene expression levels of connexin 43 and/or prolyl hydroxylase domain-containing protein 3 and connexin 37, and
wherein the subject is shown to be suffering from incidence of dementia or to have a risk of onset of dementia based on comparison of each ratio with a pre-determined cutoff value, or with a control value calculated in a similar manner for a sample derived from a healthy individual.

2. The method according to claim 1, wherein the sample is blood.

3. The method according to claim 1, wherein the dementia is Alzheimer's-type dementia.

4. A kit for determining whether there is incidence of dementia or whether there is a risk of onset of dementia,
wherein the kit comprises:
a reagent that measures gene expression level of connexin 37 in a sample derived from a subject; and
a reagent(s) that measures gene expression level(s) of connexin 43 and/or prolyl hydroxylase domain-containing protein 3 in the sample, and
wherein each reagent comprises: a primer that amplifies all or part of a base sequence of a gene or a complementary base sequence thereto; and/or a probe that hybridizes with all or part of the base sequence of the gene or the complementary base sequence thereto.

5. The kit according to claim 4, wherein each of the primer and the probe is a polynucleotide or a fragment thereof shown in any of (1) to (6):
(1) a fragment consisting of 15 or more consecutive nucleotides selected from a polynucleotide consisting of a base sequence according to SEQ ID NO:1, 3 or 5;
(2) a polynucleotide fragment having deletion, substitution, addition, or insertion of 1 or 2 bases in a base sequence of the polynucleotide fragment of (1);
(3) a fragment comprising 15 or more consecutive nucleotides selected from a polynucleotide consisting of a base sequence according to SEQ ID NO:1, 3 or 5;
(4) a polynucleotide fragment having deletion, substitution, addition, or insertion of 1 or 2 bases in a base sequence of the polynucleotide fragment of (3);
(5) a polynucleotide that hybridizes under highly stringent conditions with a fragment sequence comprising 15 or more consecutive nucleotides selected from a base sequence according to SEQ ID NO:1, 3, or 5; and
(6) a polynucleotide consisting of a base sequence complementary to the base sequence of the polynucleotide or a fragment thereof of (1) to (5).

6. A kit for determining whether there is incidence of dementia or whether there is a risk of onset of dementia,
wherein the kit comprises:
a binding molecule for connexin 37 in a sample derived from a subject; and
a binding molecule for connexin 43 and/or a binding molecule for prolyl hydroxylase domain-containing protein 3 in the sample, and
wherein each binding molecule comprises an antibody or an active fragment thereof, or an aptamer, that binds to a target protein.

7. A device that determines whether there is incidence of dementia or whether there is a risk of onset of dementia,
wherein the device comprises:
a reagent that measures gene expression level of connexin 37 in a sample derived from a subject; and
a reagent(s) that measures gene expression level(s) of connexin 43 and/or prolyl hydroxylase domain-containing protein 3 in the sample, and
wherein each reagent comprises: a primer that amplifies all or part of a base sequence of a gene or a complementary base sequence thereto; and/or a probe that hybridizes with all or part of the base sequence of the gene or the complementary base sequence thereto.

8. The device according to claim 7, wherein each of the primer and the probe is a polynucleotide or a fragment thereof shown in any of (1) to (6):
(1) a fragment consisting of 15 or more consecutive nucleotides selected from a polynucleotide consisting of a base sequence according to SEQ ID NO:1, 3 or 5;
(2) a polynucleotide fragment having deletion, substitution, addition, or insertion of 1 or 2 bases in a base sequence of the polynucleotide fragment of (1);
(3) a fragment comprising 15 or more consecutive nucleotides selected from a polynucleotide consisting of a base sequence according to SEQ ID NO:1, 3 or 5;
(4) a polynucleotide fragment having deletion, substitution, addition, or insertion of 1 or 2 bases in a base sequence of the polynucleotide fragment of (3);
(5) a polynucleotide that hybridizes under highly stringent conditions with a fragment sequence comprising 15 or more consecutive nucleotides selected from a base sequence according to SEQ ID NO:1, 3, or 5; and
(6) a polynucleotide consisting of a base sequence complementary to the base sequence of the polynucleotide or a fragment thereof of (1) to (5).

9. A device for determining whether there is incidence of dementia or whether there is a risk of onset of dementia,
wherein the device comprises:
a binding molecule for connexin 37 in a sample derived from a subject; and
a binding molecule for connexin 43 and/or a binding molecule for prolyl hydroxylase domain-containing protein 3 in the sample, and
wherein each binding molecule comprises an antibody or an active fragment thereof, or an aptamer, that binds to a target protein.

10. A biomarker for determining whether there is incidence of dementia or whether there is a risk of onset of dementia, wherein the biomarker is any one selected from a group consisting of (a) to (d):
(a) polynucleotides consisting of gene sequences for connexin 37 or a fragment thereof and connexin 43 or a fragment thereof;
(b) polynucleotides consisting of gene sequences for connexin 37 or a fragment thereof and prolyl hydroxylase domain-containing protein 3 or a fragment thereof;
(c) polypeptides consisting of connexin 37 or a fragment thereof, and connexin 43 or a fragment thereof; and
(d) polypeptides consisting of connexin 37 or a fragment thereof, and prolyl hydroxylase domain-containing protein 3 or a fragment thereof.
